(19) United States
(12) Patent Application Publication
(10) Pub. No.: US 2026/0335482 A1
(43) Pub. Date: Oct. 29, 2026

(54) METHODS AND SYSTEMS FOR STERILIZATION

(71) Applicant: Regeneron Pharmaceuticals, Inc., Tarrytown, NY (US)

(72) Inventor: Dustin BLODGETT, Altamont, NY (US)

(73) Assignee: Regeneron Pharmaceuticals, Inc., Tarrytown, NY (US)

(21) Appl. No.: 19/660,610

(22) Filed: Apr. 28, 2026

Related U.S. Application Data
(60) Provisional application No. 63/796,157, filed on Apr. 28, 2025.

(51) Int. Cl. A61L 2/208; A61L 103/15

(52) U.S. Cl. CPC A61L 2/208; A61L 2103/15; A61L 2202/15

(57) ABSTRACT

Embodiments of the present disclosure relate to systems and methods for the application of vaporized chemicals in sterilization procedures, and systems and methods for controlling (e.g., distributing, moving, or exhausting) such chemicals. For example, embodiments of the present disclosure relate to sterilization methods for sterilization loads having specific properties, including a higher percentage of liquid in the sterilization load, compared to conventional sterilization loads.

### CROSS-REFERENCE TO RELATED APPLICATION

This application claims the benefit of priority to U.S. Provisional Patent Application No. 63/796,157, filed on Apr. 28, 2025, which is hereby incorporated by reference in its entirety.

### FIELD OF THE DISCLOSURE

Various embodiments of the present disclosure relate to sterilization systems and methods for sterilization. More specifically, some embodiments of the present disclosure relate to systems and methods for chemical sterilization (e.g., moist chemical sterilization) of medical products, including terminal sterilization of drug delivery devices (e.g., pre-filled and packaged syringes) using vaporized sterilant. Additionally, embodiments of the present disclosure relate to systems and methods for monitoring and controlling environments and/or conditions within a sterilization apparatus or process.

### INTRODUCTION

Chemical sterilization processes, such as processes using ethylene oxide, vaporized hydrogen peroxide, vaporized peracetic acid, and the like, offer many advantages, such as the ability to sterilize a load at relatively low temperatures (e.g., at less than 50 °C), and without the need to enter a deep vacuum (e.g., without decreasing a pressure to below 100 millibars). Such sterilization processes may be particularly useful in the sterilization of medical devices and medical products that are sensitive to extreme temperatures and/or pressures.

Processes using chemical sterilants include steps to ensure that the sterilant reaches all parts of a load that need to be sterilized, and that after sterilization has taken place, the sterilant is removed from the load to a degree that ensures the safety and efficacy of any products that were sterilized. Additionally, sterilization processes may benefit from improvements that reduce the time and resources required to sterilize and aerate/dry a load.

In particular, the use of vaporized chemicals, such as vaporized hydrogen peroxide, may raise certain challenges. The distribution of vaporized sterilant in a sterilization system, its behavior (e.g., condensation, evaporation, etc.), and its interaction with a sterilization load (e.g., adsorption to materials of the load), may affect its efficacy and how easily it may be removed from the load. Conventional sterilization processes may be less effective for some sterilization loads. For example, material and physical properties of a sterilization load may impact the distribution and movement of vaporized sterilant.

### SUMMARY

Aspects of the present disclosure may be directed to a sterilization method comprising conducting one or more primary sterilant exposures, conducting one or more secondary sterilant exposures, and aerating a sterilization chamber. Each primary sterilant exposure may include adjusting a pressure in the sterilization chamber to a pressure of at least 500 millibars, injecting at least 125 of vaporized hydrogen peroxide into the sterilization chamber, and injecting a first amount of distribution gas, thereby raising the pressure of the sterilization chamber to a pressure of at least 900 millibar. Each secondary sterilant exposure may include adjusting the pressure in the sterilization chamber to a pressure of at least 500 millibars, injecting at least 50 grams of vaporized hydrogen peroxide into the sterilization chamber, and injecting a second amount of distribution gas, thereby raising the pressure of the sterilization chamber to a pressure of at least 900 millibar. Aerating the sterilization chamber may include adjusting a pressure within the sterilization chamber to a pressure of 450 millibars to 500 millibars, and adding an aeration gas to the sterilization chamber, thereby raising the pressure of the sterilization chamber to a pressure of 700 millibars to 1000 millibars.

In some aspects of the present disclosure, each primary sterilant exposure may include injecting at least 150 grams of vaporized hydrogen peroxide into the sterilization chamber. Each secondary sterilant exposure may include injecting at least 75 grams of vaporized hydrogen peroxide into the sterilization chamber. Sterilization methods may include conducting one or more tertiary sterilant exposures. Each tertiary sterilant exposure may include adjusting the pressure in the sterilization chamber to a pressure of at least 600 millibars, injecting at least 50 grams of vaporized hydrogen peroxide into the sterilization chamber, and injecting a third amount of distribution gas, thereby raising the pressure of the sterilization chamber to a pressure of at least 900 millibar. The one or more tertiary sterilant exposures may include at least five tertiary sterilant exposures. Each tertiary sterilant exposure may further include, after injecting the third amount of distribution gas, maintaining a distribution hold for at least 5 minutes. The one or more primary sterilant exposures may include at least two primary sterilant exposures. The one or more secondary sterilant exposures may include at least three secondary sterilant exposures. Each primary sterilant exposure may further include, after injecting the first amount of distribution gas, maintaining a distribution hold for at least 1 minute.

Aspects of the present disclosure may be directed to a sterilization method comprising conditioning a sterilization apparatus comprising a sterilization chamber, conducting one or more primary sterilant exposures, conducting one or more secondary exposures, and conducting one or more tertiary sterilant exposures. Conditioning the sterilization apparatus may include at least five conditioning pulses. Each conditioning pulse may include adjusting a pressure in the sterilization chamber to a conditioning pressure and adding a conditioning gas to the sterilization chamber, thereby raising the pressure of the sterilization chamber to a pressure that is at least 150 millibar greater than the conditioning pressure. The conditioning pressure may be 450 millibars to 650 millibars. Each primary sterilant exposure may include adjusting a pressure in the sterilization chamber to a first sterilization pressure, injecting at least 125 grams of vaporized hydrogen peroxide into the sterilization chamber, and injecting a first amount of distribution gas, thereby raising the pressure of the sterilization chamber to the first transition pressure. The first transition pressure may be at least 400 millibar greater than the first sterilization pressure. Each secondary sterilant exposure may include adjusting the pressure in the sterilization chamber to a second sterilization pressure, injecting at least 50 grams of vaporized hydrogen peroxide into the sterilization chamber, injecting a second amount of distribution gas, thereby raising the pressure of the sterilization chamber to a second transition pressure. The second transition pressure may be at least 400 millibar greater than the second sterilization pressure. Each tertiary sterilant exposure may include adjusting the pressure in the sterilization chamber to a third sterilization pressure, injecting at least 50 grams of vaporized hydrogen peroxide into the sterilization chamber, and injecting a third amount of distribution gas, thereby raising the pressure of the sterilization chamber to a third transition pressure. The third transition pressure may be at least 400 millibar greater than the third sterilization pressure.

In some aspects of the present disclosure, conditioning the sterilization apparatus includes at least ten conditioning pulses. The total amount of vaporized sterilant injected into the sterilization chamber during the one or more primary sterilant exposures, the one or more secondary sterilant exposures, and the one or more tertiary sterilant exposures may be greater than or equal to 800 grams. The one or more primary sterilant exposures may include at least 2 primary sterilant exposures. The one or more secondary sterilant exposures may include at least 3 secondary sterilant exposures. The one or more tertiary sterilant exposures may include at least 7 tertiary sterilant exposures. Sterilization methods may further include aerating the sterilization chamber. Aerating the sterilization chamber may include adjusting a pressure within the sterilization chamber to an aeration pressure, and adding an aeration gas to the sterilization chamber, thereby raising the pressure of the sterilization chamber to an exhaust pressure. The aeration pressure may be at least 400 millibars. The exhaust pressure may be at least 700 millibars. The aeration pressure may be a first aeration pressure. The first aeration pressure may be at least 480 millibars. The exhaust pressure may be a first exhaust pressure. The first exhaust pressure may be at least 900 millibars. Aerating the sterilization chamber may further include adjusting a pressure in the sterilization chamber to a second aeration pressure over an exhaust time. The second aeration pressure may be 400 millibars to 550 millibars. The exhaust time may be at least 25 minutes. Aerating the sterilization chamber may further include adding an aeration gas to the sterilization chamber, thereby raising the pressure of the sterilization chamber to a second exhaust pressure. The second exhaust pressure may be at least 900 millibars. The aeration gas, the distribution gas, and the conditioning gas may each have a dew point that is less than or equal to −10 °C.

Aspects of the present disclosure may be directed to a sterilization method comprising positioning a sterilization load in a sterilization chamber, conducting one or more sterilant exposures, and aerating the sterilization chamber. A ratio of a liquid mass in the sterilization load to a total mass of the sterilization load may be about 0.005 to about 0.02. Each sterilant exposure may include adjusting a pressure in the sterilization chamber to a pressure of at least 500 millibars, injecting at least 125 grams of vaporized hydrogen peroxide into the sterilization chamber, and injecting a first amount of distribution gas, thereby raising the pressure of the sterilization chamber to a pressure of at least 850 millibar. Aerating the sterilization chamber may include adjusting a pressure within the sterilization chamber to a pressure of at least 400 millibars, and adding an aeration gas to the sterilization chamber, thereby raising the pressure of the sterilization chamber to a pressure of at least 700 millibars.

In some aspects of the present disclosure, a ratio of liquid volume in the sterilization load to a total volume of the sterilization load is about 0.0005 to about 0.001. The sterilization load may include a plurality of pre-filled delivery devices, and each pre-filled delivery device may include at least 0.15 grams of liquid.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure. Where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

There are many inventions described and illustrated herein. The described inventions are neither limited to any single aspect nor embodiment thereof, nor to any particular combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1A is a schematic drawing of an exemplary sterilization system that may be used for sterilization of medical products, according to aspects of the present disclosure.

FIG. 1B is a schematic drawing of an exemplary sterilization system that may be used for sterilization of medical products, according to aspects of the present disclosure.

FIG. 1C is a schematic drawing showing an expanded view of a portion of the sterilization system shown in FIG. 1A, according to aspects of the present disclosure.

FIG. 2 is a flow diagram of steps in an exemplary method of sterilizing medical products using vaporized chemicals, according to aspects of the present disclosure.

FIG. 3 is a flow diagram of steps in an exemplary method of conditioning a sterilization apparatus, according to aspects of the present disclosure.

FIGS. 4A and 4B are flow diagrams of steps in an exemplary method of exposing a sterilization load to vaporized sterilant, according to aspects of the present disclosure.

FIG. 5 is a flow diagram of steps in an exemplary method of aerating a sterilization apparatus, according to aspects of the present disclosure.

FIG. 6 is a plot showing a relationship between pressure and movement of a plunger in a prefilled syringe, according to aspects of the present disclosure.

FIG. 7 is a plot of pressure and temperature within an exemplary sterilization chamber during an exemplary sterilization method, according to aspects of the present disclosure.

### DETAILED DESCRIPTION

As used herein, the terms “comprises,” “comprising,” “include,” “have,” “with,” or any other variation thereof, are intended to cover a non-exclusive inclusion, such that a process, method, article, or apparatus that comprises a list of elements need not include only those elements, but may include other elements not expressly listed or inherent to such process, method, article, or apparatus. The term “exemplary” is used in the sense of “example,” rather than “ideal.” Any implementation described herein as exemplary is not to be construed as preferred or advantageous over other implementations. Further, the terms “first,” “second,” and the like, herein do not denote any order, quantity, or importance, but rather are used to distinguish one element from another. Similarly, terms of relative orientation, such as “front side, “top side,” “back side,” “bottom side,” “upper,” “lower,” etc. are referenced relative to the described figures.

As used herein, the terms “about” and “approximately” are meant to account for possible variation of ±10% in a stated numeric value. All measurements reported herein are understood to be modified by the term “about,” or the term “approximately,” whether or not those terms are explicitly used, unless explicitly stated otherwise. As used herein, the singular forms “a,” “an,” and “the” include plural referents unless the context clearly dictates otherwise. Moreover, in the claims, values, limits, and/or ranges means the value, limit, and/or range +/−10%.

As used in the present disclosure, the term “sterilization” refers to achieving a level of sterility appropriate for a formulated drug substance or drug product for commercial distribution and use. Such a level of sterility may be defined in, for example, regulatory guidelines or regulations, such as guidelines released by the U.S. Food and Drug Administration. In some embodiments, such a level of sterility may include, for example, a 6-log reduction in microbial populations of biological indicators placed on an outside or inside surface of a drug product (e.g., an outside surface of a syringe or an inside surface of a blister pack). In other embodiments, such a level of sterility may include, for example, a 9-log or 12-log reduction in microbial populations of biological indicators. Sterilization refers to achieving such an appropriate level of sterility while also achieving a sufficiently low level of residual sterilizing chemicals (e.g., vaporized hydrogen peroxide, ethylene oxide, etc.) for commercial distribution and use. Such a low level of residual sterilizing chemical may also be defined in regulatory guidelines or regulations.

As used in the present disclosure, the term “terminal sterilization” refers to the sterilization of a drug product in a container or packaging, such as in a primary packaging component, or in both primary and secondary packaging components, suitable for commercial distribution and use.

As used in the present disclosure, the term “medical product” refers to a product for medical use on a living animal. The term “medical product” includes, for example, drug products, formulated drug substances, medical implants, medical instruments, or combinations thereof. For example, the term “medical product” may refer to a syringe containing a formulated drug substance, such as a parenteral or an ophthalmic syringe. Other exemplary medical products include, e.g., suppository applicators and medication, transdermal drug delivery devices, medical implants, needles, cannulas, medical instruments, and any other product requiring sterilization prior to an intended medical use.

As used in the present disclosure, the term “formulated drug substance” refers to a composition containing at least one active ingredient (e.g., a small molecule, a protein, a nucleic acid, or a gene therapy medicament) and one or more excipients, prepared for medical distribution and use. A formulated drug substance may include fillers, coloring agents, and other active or inactive ingredients. As used in the present disclosure, the term “drug product” refers to a dosage form that contains a formulated drug substance, such as a finished dosage form for an active ingredient. A drug product may include packaging for commercial distribution or use, such as a bottle, vial, or syringe.

As used in the present disclosure, the term “vaporized sterilant” refers to a sterilant that has been converted into a substance that may be diffused or suspended in air. In some instances, a vaporized sterilant may be a sterilant that has been combined with water and then converted into a substance that may be diffused or suspended in air. As used in the present disclosure, the term “fluid” refers to a liquid, semi-liquid, vapor, or gas including oxygen, hydrogen, nitrogen, or a combination thereof.

Embodiments of the present disclosure relate to systems and methods for the use of vaporized sterilants in sterilization processes, such as processes for sterilizing medical products. For example, embodiments of the present disclosure may relate to systems and methods for the terminal sterilization of medical products using vaporized hydrogen peroxide (VHP). More particularly, embodiments of the present disclosure may relate to, e.g., systems and methods for the terminal sterilization of medical products, such as pre-filled syringes (PFS).

Some aspects of the present disclosure may be used in combination with one or more aspects of WIPO Publication No. WO 2018/182929, filed Mar. 6, 2018, and WIPO Publication No. WO 2022/217192, filed Mar. 30, 2022, each of which is incorporated by reference in its entirety.

Medical products included in a sterilization load of a terminal sterilization process may be positioned in packaging. For example, a delivery device may be filled with a medicament and packaged in a container, envelope, blister pack, or other packaging structures. Containers, envelopes, blister packs, or other packaging structures may include a plurality of pre-filled delivery devices. Examples of packaging structures that may be used with the systems and methods described in this disclosure include those disclosed in U.S. Pat. No. 11,160,918, filed Jul. 29, 2020, U.S. Patent Publication No. 2023/0248898, filed Feb. 3, 2023, and U.S. Patent Publication No. 2024/0416028, filed Jun. 14, 2024, each of which is incorporated by reference in its entirety.

Terminal sterilization of packaged medical products (e.g., pre-filled delivery devices) may require sterilizing agents to traverse several layers of packaging materials, container materials, and/or labels, to effectively sterilize all aspects of a load, and to be appropriately removed from all aspects of the load. In some cases, packaging may include semi-permeable materials, which are permeable for sterilizing agents in a particular phase (e.g., vapor). For example, packaging may include a blister pack comprising a semi-permeable material, and plurality of medical products, prefilled with a liquid medicament, disposed in the blister pack.

Packaging for medical products may resist penetration of sterilization materials, and/or may be sensitive to temperature and pressure changes caused by sterilization. For example, medical products may be packaged in a plastic ‘blister’ configured to house the syringe and restrict it from movement. Such a blister may be only somewhat permeable to sterilization materials, and/or may be sensitive to changes in pressure.

In some aspects of the present disclosure, after delivery devices are placed into formed blisters, a blister lidding may be affixed to the blister to seal the blister. The blister lidding may include pre-applied heat-activated adhesive, where the adhesive is configured to seal the lidding to the body of the blister. The blister lidding may be rolled over the one or more blister cavities of a blister pack, and heat-sealed around a perimeter of the pack, or around perimeters of the individual blister cavities. The blister lidding may comprise a flashspun high-density polyethylene polymer, such as, for example, those sold under the Tyvek tradename. As used herein, Tyvek may refer to a flashspun high-density semi-permeable polyethylene polymer.

In the case of some types of sterilization, such as terminal sterilization, sterilizing agents may need to traverse one or more layers of packaging, to sterilize the exterior of each medical product as well as the interior of packaging elements. Sterilizing agents may also need to be successfully removed through one or more layers of packaging, to avoid remaining as residue on a medical product.

As described herein, the characteristics of a sterilization load may affect distribution and movement of vaporized sterilant during a sterilization process. As a result, conventional sterilization processes may be ineffective for sterilization loads including some arrangements of packaged drug delivery devices. For example, sterilization loads including packaged delivery devices pre-filled with a liquid medicament may be insufficiently sterilized during conventional sterilization methods. Characteristics of a sterilization load that may affect distribution and movement of vaporized sterilant during a sterilization process include a mass of the sterilization load, a ratio of a mass of the liquid medicament in the load to a total mass of the load, a volume of the delivery device, a volume of the packaging, one or more dimensions of the packaging, a material composition of the medicament, a material composition of the delivery device, and/or a material composition of the packaging.

Methods of the present disclosure may be particularly effective when used with sterilization loads having particular, weight, volume, and/or other physical characteristics. Such sterilization loads may include a greater amount of liquid compared to conventional sterilization loads. For example, methods of the present disclosure may be particularly effective when used with sterilization loads that include a greater mass of liquid relative to a total mass of the sterilization load, compared to conventional sterilization loads. Sterilization loads for which the methods of the present disclosure are particularly effective may be referred to as liquid heavy sterilization loads.

A liquid heavy sterilization load may include one or more packages (e.g., one or more blister packs), where each package includes one or more pre-filled delivery devices. Each package in a liquid heavy sterilization load may include at least about 0.15 grams of liquid mass, such as, for example, at least about 0.17 grams, at least about 0.18 grams, at least about 0.19 grams, at least about 0.20 grams, at least about 0.21 grams, at least about 0.22 grams, at least about 0.23 grams, at least about 0.24 grams, at least about 0.25 grams, about 0.15 grams to about 0.25 grams, about 0.17 grams to about 0.25 grams, about 0.19 grams to about 0.25 grams, about 0.21 grams to about 0.25 grams, about 0.15 grams to about 0.23 grams, about 0.17 grams to about 0.23 grams, about 0.19 grams to about 0.23 grams, about 0.21 grams to about 0.23 grams, about 0.15 grams to about 0.21 grams, about 0.17 grams to about 0.21 grams, about 0.19 grams to about 0.21 grams, about 0.15 grams to about 0.19 grams, about 0.17 grams to about 0.19 grams, or about 0.15 grams to about 0.17 grams.

A liquid heavy sterilization load may include at least about 500 pre-filled delivery devices, such as, for example, at least 550 pre-filled delivery devices, at least 600 pre-filled delivery devices, at least 650 pre-filled delivery devices, at least 700 pre-filled delivery devices, at least 750 pre-filled delivery devices, at least 800 pre-filled delivery devices, at least 850 pre-filled delivery devices, at least 900 pre-filled delivery devices, at least 1000 pre-filled delivery devices, at least 1500 pre-filled delivery devices, at least 2000 pre-filled delivery devices, at least 3000 pre-filled delivery devices, at least 4000 pre-filled delivery devices, at least 5000 pre-filled delivery devices, at least 6000 pre-filled delivery devices, at least 7000 pre-filled delivery devices, at least 8000 pre-filled delivery devices, at least 9000 pre-filled delivery devices, at least 10,000 pre-filled delivery devices, at least 11,000 pre-filled delivery devices, at least 12,000 pre-filled delivery devices, at least 13,000 pre-filled delivery devices, at least 14,000 pre-filled delivery devices, or at least 15,000 pre-filled delivery devices.

A liquid heavy sterilization load may include a total liquid mass of at least about 100 grams, such as, for example, at least about 105 grams, at least about 115 grams, at least about 125 grams, at least about 135 grams, at least about 145 grams, at least about 150 grams, at least about 1750 grams, at least about 1800 grams, at least about 1850 grams, at least about 1900 grams, at least about 1950 grams, 2000 grams, at least about 2050 grams, at least about 2100 grams, at least about 2150 grams, at least about 2200 grams, at least about 2250 grams, at least about 2300 grams, at least about 2350 grams, at least about 2450 grams, at least about 2500 grams, at least about 2550 grams, 100 grams to about 150 grams, about 105 grams to about 150 grams, about 110 grams to about 150 grams, about 115 grams to about 150 grams, about 120 grams to about 150 grams, about 125 grams to about 150 grams, about 130 grams to about 150 grams, about 135 grams to about 150 grams, about 140 grams to about 150 grams, about 145 grams to about 150 grams, about 100 grams to about 145 grams, about 105 grams to about 145 grams, about 110 grams to about 145 grams, about 115 grams to about 145 grams, about 120 grams to about 145 grams, about 125 grams to about 145 grams, about 130 grams to about 145 grams, about 135 grams to about 145 grams, about 140 grams to about 145 grams, about 100 grams to about 140 grams, about 105 grams to about 140 grams, about 110 grams to about 140 grams, about 115 grams to about 140 grams, about 120 grams to about 140 grams, about 125 grams to about 140 grams, about 130 grams to about 140 grams, about 135 grams to about 140 grams, about 100 grams to about 135 grams, about 105 grams to about 135 grams, about 110 grams to about 135 grams, about 115 grams to about 135 grams, about 120 grams to about 135 grams, about 125 grams to about 135 grams, about 130 grams to about 135 grams, about 100 grams to about 130 grams, about 105 grams to about 130 grams, about 110 grams to about 130 grams, about 115 grams to about 130 grams, about 120 grams to about 130 grams, about 125 grams to about 130 grams, about 100 grams to about 125 grams, about 105 grams to about 125 grams, about 110 grams to about 125 grams, about 115 grams to about 125 grams, about 120 grams to about 125 grams, about 100 grams to about 120 grams, about 105 grams to about 120 grams, about 110 grams to about 120 grams, about 115 grams to about 120 grams, about 100 grams to about 115 grams, about 105 grams to about 115 grams, about 110 grams to about 115 grams, about 100 grams to about 110 grams, about 105 grams to about 110 grams, about 100 grams to about 105 grams, about 1750 grams to about 2550 grams, about 1850 grams to about 2550 grams, about 1950 grams to about 2550 grams, about 2050 grams to about 2550 grams, about 2150 grams to about 2550 grams, about 2250 grams to about 2550 grams, about 2350 grams to about 2550 grams, about 2450 grams to about 2550 grams, about 1750 grams to about 2450 grams, about 1850 grams to about 2450 grams, about 1950 grams to about 2450 grams, about 2050 grams to about 2450 grams, about 2150 grams to about 2450 grams, about 2250 grams to about 2450 grams, about 2350 grams to about 2450 grams, about 1750 grams to about 2350 grams, about 1850 grams to about 2350 grams, about 1950 grams to about 2350 grams, about 2050 grams to about 2350 grams, about 2150 grams to about 2350 grams, about 2250 grams to about 2350 grams, about 1750 grams to about 2250 grams, about 1850 grams to about 2250 grams, about 1950 grams to about 2250 grams, about 2050 grams to about 2250 grams, about 2150 grams to about 2250 grams, about 1750 grams to about 2150 grams, about 1850 grams to about 2150 grams, about 1950 grams to about 2150 grams, about 2050 grams to about 2150 grams, about 1750 grams to about 2050 grams, about 1850 grams to about 2050 grams, about 1950 grams to about 2050 grams, about 1750 grams to about 1950 grams, about 1850 grams to about 1950 grams, or about 1750 grams to about 1850 grams.

Each pre-filled delivery device of a liquid heavy sterilization load may include a total interior volume of at least about 0.3 mL, such as, for example, at least about 0.4 mL, at least about 0.5 mL, at least about 0.6 mL, at least about 0.7 mL, at least about 0.8 mL, at least about 0.9 mL, about 0.3 mL, about 0.4 mL, about 0.5 mL about 0.6 mL, about 0.7 mL, about 0.8 mL, or about 0.9 mL.

A liquid heavy sterilization load may include at least about 0.15 mL of liquid per pre-filled delivery device, such as, for example, at least about 0.17 mL, at least about 0.18 mL, at least about 0.19 mL, at least about 0.20 mL, at least about 0.21 mL, at least about 0.22 mL, at least about 0.23 mL, at least about 0.24 mL, at least about 0.25 mL, about 0.15 mL to about 0.25 mL, about 0.17 mL to about 0.25 mL, about 0.19 mL to about 0.25 mL, about 0.21 mL to about 0.25 mL, about 0.15 mL to about 0.23 mL, about 0.17 mL to about 0.23 mL, about 0.19 mL to about 0.23 mL, about 0.21 mL to about 0.23 mL, about 0.15 mL to about 0.21 mL, about 0.17 mL to about 0.21 mL, about 0.19 mL to about 0.21 mL, about 0.15 mL to about 0.19 mL, about 0.17 mL to about 0.19 mL, or about 0.15 mL to about 0.17 mL.

After each delivery device of the liquid heavy sterilization load is pre-filled, the delivery device may include an inert or nonreactive gas such as, air, nitrogen, or another inert or nonreactive gas. The inert or nonreactive gas in the pre-filled delivery device may be referred to as a bubble. The pre-filled delivery device may include a bubble having a volume of about 0.02 mL to about 0.1 mL, such as, for example, about 0.02 mL, about 0.03 mL, about 0.04 mL, about 0.05 mL, about 0.06 mL, about 0.07 mL, about 0.08 mL, about 0.09 mL, or about 0.1 mL.

A liquid heavy sterilization load may include at least 100 mL of liquid, such as, for example, at least about 105 mL, at least about 115 mL, at least about 125 mL, at least about 135 mL, at least about 145 mL, at least about 150 mL, at least about 1750 mL, at least about 1800 mL, at least about 1850 mL, at least about 1900 mL, at least about 1950 mL, 2000 mL, at least about 2050 mL, at least about 2100 mL, at least about 2150 mL, at least about 2200 mL, at least about 2250 mL, at least about 2300 mL, at least about 2350 mL, at least about 2450 mL, at least about 2500 mL, at least about 2550 mL, 100 mL to about 150 mL, about 105 mL to about 150 mL, about 110 mL to about 150 mL, about 115 mL to about 150 mL, about 120 mL to about 150 mL, about 125 mL to about 150 mL, about 130 mL to about 150 mL, about 135 mL to about 150 mL, about 140 mL to about 150 mL, about 145 mL to about 150 mL, about 100 mL to about 145 mL, about 105 mL to about 145 mL, about 110 mL to about 145 mL, about 115 mL to about 145 mL, about 120 mL to about 145 mL, about 125 mL to about 145 mL, about 130 mL to about 145 mL, about 135 mL to about 145 mL, about 140 mL to about 145 mL, about 100 mL to about 140 mL, about 105 mL to about 140 mL, about 110 mL to about 140 mL, about 115 mL to about 140 mL, about 120 mL to about 140 mL, about 125 mL to about 140 mL, about 130 mL to about 140 mL, about 135 mL to about 140 mL, about 100 mL to about 135 mL, about 105 mL to about 135 mL, about 110 mL to about 135 mL, about 115 mL to about 135 mL, about 120 mL to about 135 mL, about 125 mL to about 135 mL, about 130 mL to about 135 mL, about 100 mL to about 130 mL, about 105 mL to about 130 mL, about 110 mL to about 130 mL, about 115 mL to about 130 mL, about 120 mL to about 130 mL, about 125 mL to about 130 mL, about 100 mL to about 125 mL, about 105 mL to about 125 mL, about 110 mL to about 125 mL, about 115 mL to about 125 mL, about 120 mL to about 125 mL, about 100 mL to about 120 mL, about 105 mL to about 120 mL, about 110 mL to about 120 mL, about 115 mL to about 120 mL, about 100 mL to about 115 mL, about 105 mL to about 115 mL, about 110 mL to about 115 mL, about 100 mL to about 110 mL, about 105 mL to about 110 mL, about 100 mL to about 105 mL, about 1750 mL to about 2550 mL, about 1850 mL to about 2550 mL, about 1950 mL to about 2550 mL, about 2050 mL to about 2550 mL, about 2150 mL to about 2550 mL, about 2250 mL to about 2550 mL, about 2350 mL to about 2550 mL, about 2450 mL to about 2550 mL, about 1750 mL to about 2450 mL, about 1850 mL to about 2450 mL, about 1950 mL to about 2450 mL, about 2050 mL to about 2450 mL, about 2150 mL to about 2450 mL, about 2250 mL to about 2450 mL, about 2350 mL to about 2450 mL, about 1750 mL to about 2350 mL, about 1850 mL to about 2350 mL, about 1950 mL to about 2350 mL, about 2050 mL to about 2350 mL, about 2150 mL to about 2350 mL, about 2250 mL to about 2350 mL, about 1750 mL to about 2250 mL, about 1850 mL to about 2250 mL, about 1950 mL to about 2250 mL, about 2050 mL to about 2250 mL, about 2150 mL to about 2250 mL, about 1750 mL to about 2150 mL, about 1850 mL to about 2150 mL, about 1950 mL to about 2150 mL, about 2050 mL to about 2150 mL, about 1750 mL to about 2050 mL, about 1850 mL to about 2050 mL, about 1950 mL to about 2050 mL, about 1750 mL to about 1950 mL, about 1850 mL to about 1950 mL, or about 1750 mL to about 1850 mL.

Each package in a liquid heavy sterilization load may have a length of about 135 mm to about 145 mm, such as, for example, about 135 mm, about 136 mm, about 137 mm, about 138 mm, about 139 mm, about 140 mm, about 141 mm, about 142 mm, about 143 mm, about 144 mm, or about 145 mm.

Each package in a liquid heavy sterilization load may have a width of about 70 mm to about 85 mm, about 70 mm to about 80 mm, about 70 mm to about 75 mm, about 75 mm to about 85 mm, about 75 mm to about 85 mm, or about 75 mm to about 80 mm, such as, for example, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, or about 85 mm.

Each package in a liquid heavy sterilization load may have a height of about 20 mm to about 30 mm, about 20 mm to about 25 mm, or about 25 mm to about 30 mm, such as, for example, about 20 mm, about 21 mm, about 22 mm, about 23 mm, about 24 mm, about 25 mm, about 26 mm, about 27 mm, about 28 mm, about 29 mm, or about 30 mm.

As described herein, a package within a sterilization load may comprise a flashspun high-density semi-permeable polyethylene polymer. In a liquid heavy sterilization load, the flashspun high-density semi-permeable polyethylene polymer may have a thickness of about 0.2 mm to about 1.5 mm, about 0.5 mm to about 1.5 mm, or about 1.0 to about 1.5 mm, such as, for example, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, or about 1.5 mm.

Each package in a liquid heavy sterilization load may have a volume of about 250 mL to about 300 mL, about 275 mL to about 300 mL, or about 250 mL to about 275 mL, such as for example, about 250 mL, about 255 mL, about 260 mL, about 265 mL, about 270 mL, about 275 mL, about 280 mL, about 285 mL, about 290 mL, about 295 mL, or about 300 mL.

A liquid heavy sterilization load may have a volume of about 155 L to about 3200 L, about 155 L to about 170 L, or about 2900 L to about 3000 L, such as, for example, about 155 L, about 160 L, about 165 L, about 170 L, about 2900 L, about 2950 L, about 3000 L, about 3050 L, about 3150 L, or about 3200 L.

A liquid heavy sterilization load may include a ratio of liquid mass to total load mass of about 0.005 to about 0.02, or about 0.011 to about 0.013, such as, for example, about 0.005, about 0.006, about 0.007, about 0.008, about 0.009, about 0.01, about 0.011, about 0.012, about 0.013, about 0.014, about 0.015, about 0.016, about 0.017, about 0.018, about 0.019, or about 0.2.

A liquid heavy sterilization load may include a ratio of liquid volume to total load volume of about 0.0005 to about 0.001, or about 0.0006 to about 0.00075, such as, for example, about 0.0005, about 0.00055, about 0.0006, about 0.00065, about 0.0007, about 0.00075, about 0.0008, about 0.00085, about 0.0009, about 0.00095, or about 0.001.

During a sterilization process, medical products may be located within different portions of a sterilization chamber. Throughout the sterilization process, each portion of the sterilization chamber may exhibit conditions that may differ from those in other parts of the chamber, such as temperature, pressure, water vapor concentration, humidity, or sterilant concentration. The differing conditions may affect sterilization efficacy. Maintenance of a consistent environment throughout a sterilization chamber may be beneficial to ensuring sterilization efficacy is adequate as to all parts of a load.

An environment within a sterilization chamber may change during a sterilization cycle, such as in a way that affects the movement, state, or efficacy of sterilant and/or fluids in the chamber. For example, as sterilant is added to a chamber, a pressure within the chamber may increase. The pressure within the chamber may affect the ratio of condensed sterilant to vaporized sterilant. Changes in temperature, humidity, or other environmental characteristics may also affect how sterilant within the chamber and additional sterilant added to the chamber behaves. Sterilization systems and methods that adapt to changes in the environment or climate within the sterilization chamber during a sterilization phase, to maximize efficacy of sterilization, may be beneficial. Systems and methods that adapt to changes in the environment or climate of an area to maximize aeration and drying of that area may also be beneficial.

Using a combination of a vaporized chemical sterilant (e.g., VHP) and vaporized water in an environment in which temperature and pressure may be precisely controlled may allow for specific management of the environment to maximize the sterilant's contact with a sterilization load during a sterilization phase, and/or to maximize removal of the sterilant from a load during one or more subsequent aeration or drying phases. Some embodiments of the present disclosure are related to precisely controlling one or more of temperature, pressure, humidity, exposure times, and other environmental conditions. Environmental conditions may be adjusted in any portion of the sterilizing apparatus, before, during, and/or after a sterilization process is performed with the apparatus. For example, the environment of one or more portions of the apparatus where sterilant is introduced or removed, may be maintained or controlled to be within pre-determined conditions. As such, embodiments of the present disclosure may aid in improving the introduction and/or removal of a chemical sterilant in a sterilization apparatus (e.g., between the sterilization apparatus and an exterior of the apparatus, or between portions of the apparatus).

For example, embodiments of the present disclosure may allow for improved infiltration of sterilant through one or more layers of a medical device and/or packaging, compared to conventional methods. In addition or alternatively, embodiments of the present disclosure may allow for improved exfiltration of sterilant through one or more layers of medical device and/or packaging, compared to conventional methods and systems. The improved infiltration of sterilant and/or improved exfiltration of sterilant described herein may be more pronounced when the methods and systems disclosed herein are used with liquid heavy sterilization loads.

Several characteristics of a vaporized chemical sterilant may (positively or negatively) affect the safety, efficacy, efficiency, and other aspects of sterilization processes for medical products. Chemical sterilant vapors and water vapors in an environment may adsorb to, and/or condense on, surfaces having relatively cooler temperatures within the sterilization chamber. For example, during vapor sterilization of sterilization loads including pre-filled delivery devices or medical produces, “cold spots” created by liquid medicament (e.g., a liquid with a high heat capacity), may attract vapor adsorption and/or promote surface condensation. The relative size and mass of the “cold spots” of liquid medicament may affect how chemical sterilant and water vapors adsorb on surfaces, condense on surfaces, or traverse surfaces (e.g., permeable packaging layers). Parameters of a sterilization method may be adjusted, depending on the relative sizes and distributions of liquid medicaments within the sterilization load, in order to promote improved transportation of the sterilant through the sterilization load.

Adjustments in the temperature of a sterilization chamber may generate areas of relative warmth and coolness within the environment, which may in turn affect the relative temperature of a sterilization load in an area of relative warmth or coolness. For example, heating a sterilization chamber using a temperature control jacket may result in areas of the chamber closest to the jacket (e.g., a periphery of the chamber) becoming warmer than areas farther from the jacket (e.g., a middle of the chamber). The ambient heat in warmer areas may cause parts of a sterilization load in those areas to become relatively warmer as well. Chemical sterilant vapors and water vapors may preferentially adsorb to the surfaces of areas having relatively cooler temperatures as compared to the rest of the environment (“cold spots”); thus, vaporized chemical sterilant (e.g., VHP) may not distribute evenly between areas of relative warmth and coolness. While cooler areas may be subject to more thorough exposure to sterilant, warmer areas may experience more thorough aeration and drying.

VHP may preferentially adsorb onto surfaces as compared to water vapor, due to the fact that hydrogen peroxide is more dense and less volatile than water. In some instances, hydrogen peroxide and water vapor may be adsorbing and condensing on surfaces at the same time, with hydrogen peroxide adsorbing and condensing in greater quantities and percentages as compared to the water vapor, and in closer proximity to the surfaces of the sterilization load than the water vapor. Multiple layers of adsorption may form on a single surface in a sterilization environment. In some instances, each layer of adsorption and/or condensation further away from the surface may contain less hydrogen peroxide and more water vapor, such that a gradient of hydrogen peroxide to water is formed on the surface. Hydrogen peroxide may preferentially adsorb and condense closer to the surface than water because of the thermodynamic behavior of binary mixtures of VHP and water vapor near or at saturation (e.g., a binary mixture of hydrogen peroxide and water at vapor/liquid equilibrium). Vapor/liquid equilibrium may be analogous to gas/adsorbate equilibrium for binary mixtures of VHP and water vapor in sterilization applications.

In some instances, condensed or adsorbed hydrogen peroxide may be difficult to remove from surfaces. For example, condensation of water vapor over the condensed/adsorbed hydrogen peroxide, or adsorption of water around the condensed/adsorbed hydrogen peroxide, may trap the hydrogen peroxide on the sterilized surface, or otherwise inhibit the removal of the hydrogen peroxide. This effect may be more pronounced in sterilization loads having relatively higher masses or volumes of liquid medicament (e.g., a higher ratio of mass of liquid medicament to mass of sterilization load).

Differences in pressure throughout an environment, such as a sterilization chamber, may also affect efficacy of vaporized chemical sterilant. For example, sterilization efficacy may be greater at compressed air injection points of a sterilization chamber, compared to the rest of the chamber. Without being limited by theory, this may be due to the nature of gas within the chamber at a partial vacuum. Areas local to a compressed air injection point may experience a pressure wave or pulse having greater magnitude than areas farther from a compressed air injection point while the chamber is saturated with chemical sterilant. The pressure wave may cause greater condensation of chemical sterilant in areas local to compressed air injection points. The characteristics and movement of the pressure wave may be affected by characteristics of the sterilization load. For example, sterilization loads including relatively higher masses or volumes of liquid medicament may impede or dampen pressure waves generated in the sterilization chamber, compared to sterilization loads with lower masses and volumes of liquid medicament.

In some cases where it is desired that a sterilant traverse a semi-permeable membrane of a load to sterilize an interior area or volume covered by the membrane, a delay in migration of at least a portion of sterilant through the load has been observed. For example, in sterilization loads including a semi-permeable Tyvek membrane, the hydrogen peroxide concentration within the membrane lagged, or was slower to equilibrate with, the hydrogen peroxide concentration outside of the membrane. No such delay or lag was observed with respect to water concentration. Thus, the relative strength of a sterilant may be lower for loads or portions of a load within a semi-permeable membrane (either for part or for all of a sterilization cycle) as compared to the strength of the sterilant outside of the membrane. This effect may be more pronounced in sterilization loads having relatively higher masses or volumes of liquid medicament (e.g., a higher ratio of mass of liquid medicament to mass of sterilization load). Accordingly, parameters of sterilization methods for such loads may need to be adjusted in order to ensure proper aeration, drying, and/or removal of sterilant.

The speed with which pressure increases during introduction of vaporized sterilant into a sterilization chamber may negatively affect sterilization efficacy. A pressure increase to some degree may benefit introduction of sterilant to a load, promoting adsorption of the sterilant to the load. However, excessive pressure increases when an environment is at or near a level of VHP saturation, for example, may result in aggressive condensation of the VHP, which may compromise sterilization efficacy or subsequent aeration or drying efficacy. Allowing the pressure of an environment to be held at a level where vaporized sterilant may condense over time may result in over-condensation of vaporized sterilant. Conversely, decreasing the pressure of an environment after introducing vaporized sterilant (e.g., VHP) may allow more sterilant to remain in a vapor phase, which may improve the sterilant's efficacy in migrating through semi-permeable membranes and effecting sterilization of the interior(s) of a sterilization load.

During a sterilization phase (e.g., a sterilization pulse), increases in pressure may be faster than decreases in pressure (e.g., the rate of pressure increase during a sterilization pulse may be at least 150 millibar/minute faster than the rate of pressure decrease during the same pulse). This may promote travel of the sterilant (e.g., promote travel of sterilant through one or more layers of packaging). During aeration, the reverse may be employed to promote travel of sterilant from within layers of packaging to exterior of the packaging, and through the exhaust of the sterilization apparatus. For example, the rate of pressure decrease during an aeration pulse may be at least 150 millibar/minute faster than the rate of pressure increase during the same pulse. Increased chamber temperatures may also increase the efficiency of aeration.

The saturation level of the sterilization chamber may also factor into the rate or direction of pressure adjustment. For example, pressure increases near atmospheric pressure should be avoided while the sterilization chamber is near saturation. For example, greater pressure changes may be used at lower sterilant concentrations, while large pressure changes at high sterilant concentrations may cause excess condensation, decreasing the efficiency of the sterilization.

Sterilizing agents may also need to be successfully removed from the load to avoid remaining as residue on or in a medical product. For example, in embodiments utilizing packaging including a semi-permeable membrane, traversal of the semi-permeable membrane may only be possible for sterilizing agents in a particular form, such as a vapor or gas. In some cases, stimulating mechanical movement (e.g., rocking, rotation, agitation, etc.) of some or all of a load may dislodge sterilant molecules adhered to a load, and may promote aeration and removal of a sterilant from the load. Low frequency pressure waves or acoustics generated within the sterilization chamber (for example, via a diaphragm or piston located within the chamber) may dislodge sterilant adhered to the load.

Immediately increasing the pressure following a sterilization pulse can cause unnecessary condensation and result in less efficacious aeration. Prior to aeration, the humidity of the sterilization chamber may be reduced to prevent excessive condensation. For example, the contents of the closed system may be passed through a condenser (e.g., a desiccant wheel) to reduce the humidity of the environment. In addition or alternatively, dry air may be injected, prior to, or during exhaustion, to reduce the overall humidity of the system.

The effects of cold spots or areas of relative warmth and coolness on the distribution and movement of sterilant during sterilization processes may be more pronounced in sterilization loads including relatively greater masses of liquid medicament, and/or relatively greater ratios of medicament mass to total mass of the sterilization load. Accordingly, the parameters of sterilization methods should be tailored to characteristics of the sterilization load.

Systems and methods disclosed herein may advantageously be used in improving the efficacy of sterilization, aeration, and/or drying cycles involving vaporized chemical sterilant. For example, systems and methods of the present disclosure may inject more total vaporized sterilant, and provide more sterilant exposure time to the product load, resulting in effective sterilization and aeration within a shorter total cycle duration, compared to conventional methods and systems. In particular, systems and methods disclosed herein may provide improved sterilization efficiency for liquid heavy sterilization loads.

For example, systems and methods disclosed herein may provide for full (e.g., 100%) sterilization of medical products using VHP, followed by full (e.g., 100%) removal of VHP from sterilized products. Systems and methods disclosed herein may, e.g., increase efficiency, safety, and efficacy of sterilization, and/or decrease sterilization cycle time. While aspects of the present disclosure may be described with respect to the use of VHP in terminal sterilization of PFS, the present disclosure contemplates using the techniques and systems herein for movement of VHP and other chemical sterilants in other contexts as well (e.g., sterilization of other products, cleaning areas, addition/removal of vaporized chemical to any environment, etc.).

The present disclosure also contemplates performance of “moist chemical sterilization,” by which chemical sterilization may be achieved in the presence of water vapor. Comparison of “moist chemical sterilization” to “chemical sterilization” may be analogous, in some cases, to comparison of “moist heat sterilization” to “heat sterilization.” In some instances, moist chemical sterilization may be a more effective and efficient means of achieving sterilization than chemical sterilization technology that currently exists, in the same way that “moist heat sterilization” is considered to be, in some cases, more effective and efficient than only “heat sterilization.”

“Moist chemical sterilization” may take place when environmental conditions of relatively high chemical concentration, high water vapor concentration, and high pressure (e.g., above 400 millibar) act in concert to force the chemical and water vapor to behave as a binary mixture. In order to achieve the desired relatively high chemical concentration, high water vapor concentration, and high pressure, the area to be sterilized may be saturated with a combination of water vapor and sterilizing chemical (e.g., VHP), forcing vapor to condense on surfaces of the load. Most commercially available hydrogen peroxide is available and sold as aqueous liquid mixtures in varying concentrations (e.g., 3%, 15%, 35%, 59%), and thus, vaporizing hydrogen peroxide will generally simultaneously include vaporizing water.

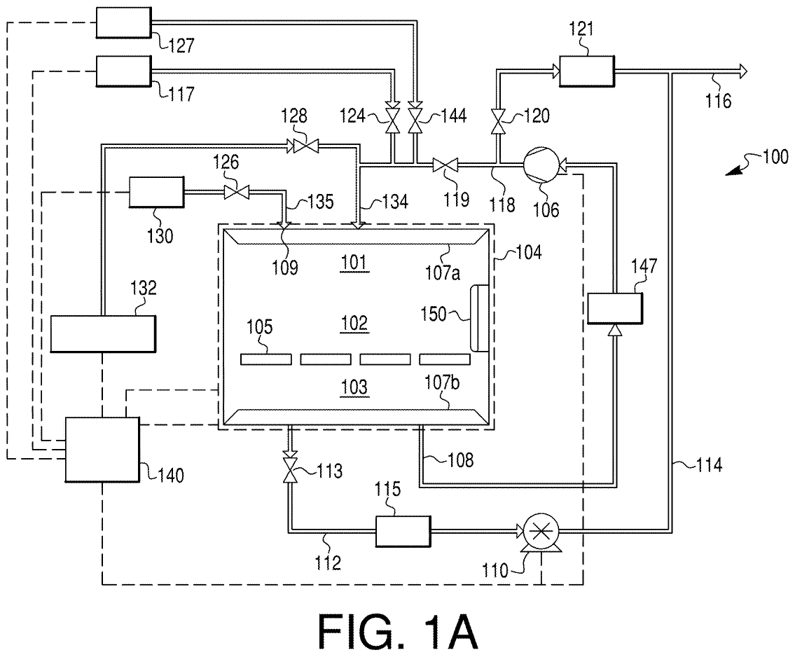

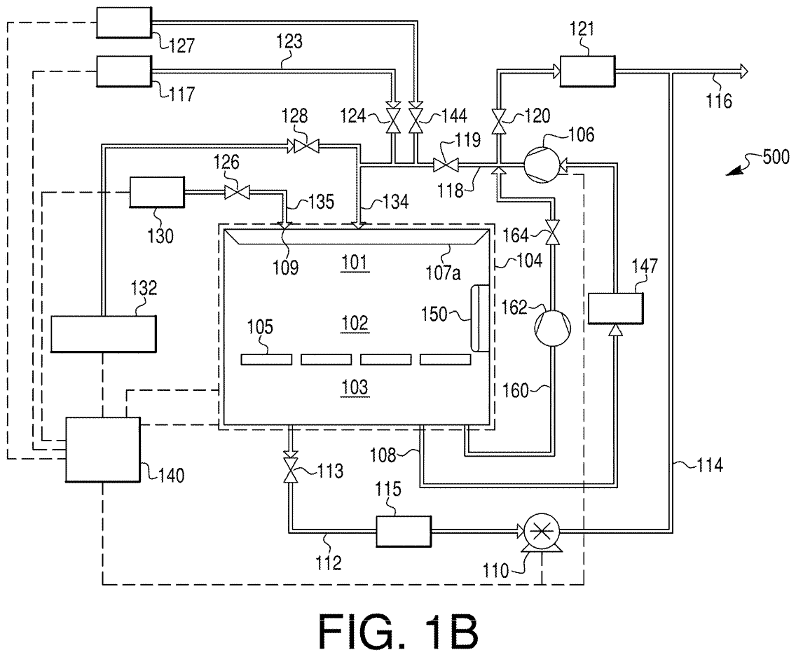

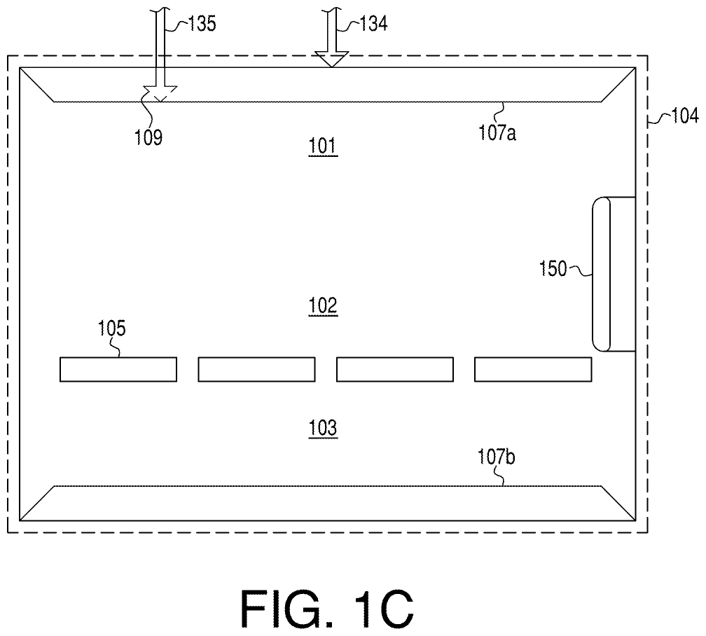

Referring now to the figures, FIG. 1A depicts in schematic form an exemplary sterilization system 100 for use in methods of the present disclosure. It is to be understood that sterilization system 100 is merely exemplary, and methods disclosed herein may be used in many other systems, environments, and/or parts thereof. Sterilization system 100 includes a sterilization chamber 102, surrounded by a temperature control jacket 104. Sterilization chamber 102 has an interior cavity, including an upper interior 101 and a lower interior 103. Sterilization chamber 102 is configured to house a sterilization load (e.g., a load including one or more products 105) for sterilization. An inlet conduit 134, fluidly connected to sterilization chamber 102, is configured to allow various fluids to enter sterilization chamber 102. The inlet conduit 134 may be connected to one or more distribution manifolds (e.g., diffusion plates, spray balls, or other structure configured to distribute a gas throughout the chamber). In the examples shown in FIGS. 1A and 1C, a first distribution manifold 107a is located near upper interior 101 and a second distribution manifold 107b is located near lower interior 103. Placement of distribution manifolds 107a, 107b on opposing sides of sterilization chamber 102 may promote even distribution of sterilant, air, or other introduced material.

Inlet conduit 134 may connect to both first distribution manifold 107a and second distribution manifold 107b. In some embodiments, second distribution manifold 107b is connected to a different inlet conduit than first distribution manifold 107a.

A second inlet conduit 135 is also fluidly connected to sterilization chamber 102, also to allow fluids to enter sterilization chamber 102 via an inlet 109. For example, ambient air from auxiliary ambient air supply 130 may be introduced into sterilization chamber 102 via inlet 109.

A blower 106 is fluidly connected to sterilization chamber 102 via a blower exit conduit 108. A blower circulation conduit 118 fluidly connects blower 106 to move fluids from blower exit conduit 108 either towards an exhaust 116, or back towards sterilization chamber 102 via inlet conduit 134. In some embodiments, blower exit conduit 108 may include, or be coupled to, a condenser 147. Condenser 147 may include a desiccate wheel or other structure configured to remove water from the fluid passing through blower exit conduit 108. An exhaust valve 120 is located between blower circulation conduit 118 and exhaust 116, and selectively closes or opens a connection between blower circulation conduit 118 and exhaust 116. A recirculation valve 119 is located between blower circulation conduit 118 and inlet conduit 134, and selectively closes or opens a connection between blower 106 (e.g., via blower circulation conduit 118) and inlet conduit 134.

Blower 106 may circulate air at a rate greater than 500 cubic feet per minute (cfm). In some embodiments, the rate of air circulation maintained by blower 106 may be less than 1000 cfm. If blower 106 is moving too much fluid, sterilant will be displaced near distribution manifolds 107a, 107b, decreasing the sterilization efficiency.

A vacuum pump 110 may be fluidly connected to sterilization chamber 102, via a vacuum conduit 112. Vacuum conduit 112 may include, or be connected to, a catalytic converter 115. A vacuum valve 113 located between sterilization chamber 102 and vacuum conduit 112, may selectively allow, partially allows, or block, flow from sterilization chamber 102 (e.g., through catalytic converter 115) to vacuum pump 110. A vacuum exhaust conduit 114 fluidly connects vacuum pump 110 to exhaust 116.

Sterilization system 100 may include several supplies of air and/or vapor from which fluid may be introduced into sterilization chamber 102 via inlet conduit 134 or inlet conduit 135. A dry air supply 127 may be configured to supply dry air to sterilization chamber 102 via inlet conduit 134. In some embodiments, dry air supply 127 is compressed dry air. A dry air valve 144 may be coupled to the fluid connection between dry air supply 127 and inlet conduit 134. The dry air valve 144 may selectively allow, partially allow, or block, flow of dry air from dry air supply 127 to sterilization chamber 102 via inlet conduit 134.

An ambient air supply 117 may be configured to supply ambient air (e.g., air comprising more humidity than dry air from dry air supply 127) to sterilization chamber 102, via inlet conduit 134. In some embodiments, ambient air may be drawn from an environment (e.g., a room) around the sterilization system 100. An ambient air valve 124 may be coupled to the fluid connection between ambient air supply 117 and inlet conduit 134. The ambient air valve 124 may selectively allow, partially allow, or block, flow of ambient air from ambient air supply 117 to sterilization chamber 102 via inlet conduit 134.

Ambient air supply 117, for example, may be any supply of air (e.g., room air, or compressed air) or other fluid external from the rest of sterilization system 100. In some embodiments, ambient air supply 117 may be a supply of “room air” surrounding sterilization system 100, which may have gone through an indoor filtration system. In some embodiments, ambient air supply 117 may include more water vapor than “room air.” In some embodiments, ambient air supply 117 may be a supply of filtered outdoor air.

A VHP injector 132, fluidly connected to inlet conduit 134, is configured to inject VHP to sterilization chamber 102 via inlet conduit 134. A VHP injector valve 128 is coupled to the fluid connection between VHP injector 132 and inlet conduit 134, and selectively allows, partially allows, or blocks flow of VHP from VHP injector 132 to sterilization chamber 102 via inlet conduit 134.

VHP injector 132 may include a supply of VHP, or VHP and vaporized water, and may be configured to inject VHP or a combination of VHP and vaporized water into sterilization chamber 102 via, e.g., inlet conduit 134. VHP injector 132 may be configured to inject vapor into sterilization chamber 102 (or inlet conduit 134) at an adjustable concentration.

Depending on the position of dry air valve 144, ambient air valve 124, and VHP injector valve 128, dry air from dry air supply 127, ambient air from ambient air supply 117, VHP from VHP injector 132, or a combination thereof, may enter sterilization chamber 102 via inlet conduit 134. For example, during conditioning, ambient air valve 124 may be in a position such that ambient air from ambient air supply 117 is blocked from entering inlet conduit 134, VHP injector valve 128 may be in a position such that VHP from VHP injector 132 is blocked from entering inlet conduit 134, and dry air valve 144 may be in a position such that dry air flows from dry air supply 127 to sterilization chamber 102, via inlet conduit 134. In such a configuration, only dry air flows to the sterilization chamber 102, allowing for faster conditioning.

In another configuration, ambient air valve 124 may be in a position such that ambient air flows from ambient air supply 117 to sterilization chamber 102 (i.e., via inlet conduit 134), VHP injector valve 128 may be in a position such that VHP flows from VHP injector 132 to sterilization chamber 102 (i.e., via inlet conduit 134), and dry air valve 144 may be in a position such that dry air from dry air supply 127 is blocked from entering inlet conduit 134. In such a configuration, ambient air may function as a binding media to carry VHP into sterilization chamber 102. Dry air or a combination of dry air and ambient air may also be used as binding media. Ambient air may be more effective as binding media than dry air. Humidity (e.g., water) from the ambient air used as binding media may be removed from sterilization chamber 102 (e.g., via condenser 147) after VHP has been introduced.

An auxiliary ambient air supply 130 fluidly connected to inlet conduit 135 may be configured to supply ambient air to sterilization chamber 102 via inlet conduit 135. An auxiliary supply valve 126 is coupled to the fluid connection between auxiliary ambient air supply 130 and inlet conduit 135, and is configured to selectively allow, partially allow, or block flow of dry air from auxiliary ambient air supply 130 to sterilization chamber 102 via inlet conduit 135.

Dry air supply 127 and auxiliary ambient air supply 130 may have the same composition or different compositions. Dry air supply 127 may be a supply of air having a relatively low humidity, such that it may be used to dry sterilization chamber 102 (e.g., a portion of sterilization chamber 102) and/or one or more of blower exit conduit 108, vacuum conduit 112, vacuum exhaust conduit 114, blower circulation conduit 118, and inlet conduit 134, 135. For example, in some embodiments, air in dry air supply 127 may include a dew point of, e.g., −10 degrees Celsius or less, −40 degrees Celsius or less, or anywhere between −10 degrees Celsius and −40 degrees Celsius. In some embodiments, dry air supply 127 may be a supply of hygienic dry air, such as air that has been sterilized or otherwise filtered to at least 0.2 microns. In some embodiments, dry air supply 127 may be a sealed supply of air. In some embodiments, dry air supply 127 may be a supply of compressed air. As described herein, conditioning gas, distribution gas, and/or aeration gas may be provided from dry air supply 127, ambient air supply 117, and/or auxiliary ambient air supply 130.

Sterilization system 100 may be configured to run sterilization cycles within sterilization chamber 102 at a variety of temperatures and pressures, and for a variety of time durations and/or time intervals. In some embodiments, the temperature(s), pressure(s), and time interval(s) at which sterilization system 100 may run sterilization cycles may be selectively and individually modified and customized. Moreover, temperature(s), pressure(s), and time intervals(s) may be adjusted during sterilization cycles, e.g., to effect improved distribution, migration, and/or removal of sterilants.

Sterilization system 100 may be configured to control the environment in the interior of sterilization chamber 102, including one or more of temperature, pressure, humidity, atmosphere, intake of fluids, and exit of fluids. Mechanisms for controlling temperature include: temperature modulation of input fluid streams and/or recirculating fluid streams, temperature modulation of portions of the sterilization chamber 102 itself, and temperature modulation of other components of the sterilization system 100 (e.g., intake conduit 134, blower exit conduit 108, vacuum pump 110, temperature control jacket 104, blower circulation conduit 118, blower 106, recirculation valve 119, ambient air supply 117, dry air supply 127, VHP injector 132, ambient air valve 124, dry air valve 144, VHP injector valve 128, and/or distribution manifolds 107a, 107b). Sterilization chamber 102 (e.g., a portion of sterilization chamber 102, such as, upper interior 101 or lower interior 103) may include, or be fluidly connected to one or more pistons 150 or diaphragm that may be actuated, inflated, deflated, or otherwise altered, to modulate the pressure of sterilization chamber 102 without introduction or removal of matter. The one or more pistons 150 or diaphragms may be configured to create pressure waves or generate low frequency pulses. These pressure waves and pulses may be used to affect (e.g., promote) condensation of sterilant on a surface of the load (e.g., products 105).

Further, sterilization system 100 may include any suitable number and location of sensors configured to sense, e.g., temperature, pressure, flow, chemical concentration, or other parameters throughout sterilization system 100, including in sterilization chamber 102, temperature control jacket 104, blower 106, vacuum pump 110, and/or any of conduits 108, 112, 114, 118, and 134. Such sensors may be configured to transmit sensed data to, e.g., a controller 140 and/or a human-machine interface.

Sterilization chamber 102 may be a sealable chamber defining an interior, including upper interior 101 and lower interior 103. Sterilization chamber 102 may be openable into an open configuration, such that one or more items, e.g., products 105, may be placed inside as a part of a load for sterilization, and may be removed subsequent to sterilization. In some embodiments, sterilization chamber 102 may have an operating orientation, e.g., such that upper interior 101 is located above lower interior 103, and such that matter may fall (e.g., under the forces of gravity) from the vicinity of upper interior 101 towards lower interior 103. Sterilization chamber 102 may have one or more delivery apparatuses to which one or more of inlet conduit 134 and inlet conduit 135 may be connected. As depicted in FIG. 1A, for example, distribution manifolds 107a, 107b are two such delivery apparatuses. Distribution manifolds 107a, 107b may be configured to disperse gas, vapor, or liquid into sterilization chamber 102 in a given configuration, such as a stream or an even spray across sterilization chamber 102. For example, first distribution manifold 107a may distribute gas, vapor, or liquid across upper interior 101 and second distribution manifold 107b may distribute gas, vapor, or liquid across lower interior 103. Inlet 109 is another such delivery apparatus. Inlet 109 may also be configured to disperse gas, vapor, or liquid into sterilization chamber 102 in a given configuration, such as a stream, or an even spray across upper interior 101, or another portion of sterilization chamber 102.

In some embodiments, a distribution manifold (e.g., first distribution manifold 107a) may be configured to disperse gas, vapor, or liquid into sterilization chamber 102 in one configuration, such as an even spray, and inlet 109 may be configured to disperse gas or vapor into sterilization chamber 102 in a different configuration, such as in a stream. In some embodiments, there may be no inlet 109, and both inlet conduits 134 and 135 may be connected to one or more distribution manifolds 107a, 107b.

Temperature control jacket 104 may be any material surrounding sterilization chamber 102, that is configured or effective to afford temperature control to the environment inside sterilization chamber 102. In some embodiments, for example, temperature control jacket 104 may be a water jacket surrounding sterilization chamber 102. In such embodiments, a temperature and/or a flow of water or other liquid through temperature control jacket 104 may be controlled by, e.g. controller 140.

Products 105 may be any item or items in a load suitable for sterilization using sterilization system 100. In some embodiments, products 105 may be medical products in primary packaging, secondary packaging, or both. In some embodiments, products 105 may be medical products having moving parts or parts otherwise sensitive to deep vacuum environments, such as environments having pressure of less than about 100 millibars. In some embodiments, products 105 may be, e.g., containers filled with a volume of formulated drug substance. For example, products 105 may be vials or PFS. In some embodiments, products 105 may include or may be covered by semi-permeable packaging, such as a semi-permeable membrane, through which vapor or gas may pass. In further embodiments, products 105 may be or include medical products sensitive to high temperatures, e.g., above 30 °C. Such medical products may include, for example, formulated drug substances or other compositions that may be sensitive to high temperatures, such as proteins (e.g., antibodies or enzymes), fragments thereof, any antigen-binding molecules, nucleic acids, blood, blood components, vaccines, allergenics, gene therapy medicaments, tissues, other biologics, etc. For example, products 105 may be packaged PFS containing a formulated drug substance that includes an antibody or an adeno-associated virus (AAV). In some embodiments, products 105 may include drug products including a large molecule, e.g., a molecular weight of 30 kDA or greater. In some embodiments, products 105 may include ingredients such as, e.g., aflibercept, alirocumab, abicipar pegol, bevacizumab, brolucizumab, conbercept, dupilumab, evolocumab, tocilizumab, certolizumab, abatacept, rituximab, infliximab, ranibizumab, sarilumab, adalimumab, anakinra, trastuzumab, pegfilgrastim, interferon beta-la, insulin glargine [rDNA origin], epoetin alpha, darbepoetin, filigrastim, golimumab, etanercept, antigen-binding fragments of any of the above, or combinations of such binding domains, such as a bispecific antibody to VEGF or angiopoietin-2, among others.

In some embodiments, products 105 may include therapeutic products for ophthalmic diseases, including for the treatment of patients with Neovascular (Wet) Age-related Macular Degeneration (AMD), Macular Edema following Retinal Vein Occlusion (RVO), Diabetic Macular Edema (DME), and Diabetic Retinopathy (DR). In particular, large molecule and small molecule antagonists of VEGF and/or ANG-2, such as aflibercept, ranibizumab, bevacizumab, conbercept, OPT-302, RTH258 (brolocizumab), abicipar pegol (a pegylated designed ankyrin repeating protein (DARPin)), RG7716, or fragments thereof and in any concentration may be included in products 105. In some embodiments, products 105 may be products for cosmetic applications or medical dermatology, such as treatment or diagnosis of allergic responses.

Blower 106 may be, for example, a blower having the capacity to forcibly draw vapor and gas from lower interior 103 of sterilization chamber 102 through blower exit conduit 108, optionally through condenser 147, and to reintroduce said vapor and gas back to upper interior 101 of sterilization chamber 102 via inlet conduit 134 (or, alternatively, to draw such vapor and gas through exhaust valve 120 and catalytic converter 121, to exhaust 116). In some embodiments, blower 106 may be external to sterilization chamber 102, as shown in FIG. 1A. In other embodiments, blower 106 may be disposed within sterilization chamber 102. In some embodiments, blower 106 may be configured to draw vapor and gas from lower interior 103 of sterilization chamber 102 and reintroduce said vapor and gas back to upper interior 101 with sufficient force to create a flow of vapor and gas from upper interior 101 to lower interior 103 of sterilization chamber 102. This flow may be termed a “turbulent flow.” In some embodiments, the force with which blower 106 may operate may be adjustable (via, for example, controller 140), such that a more turbulent (e.g., more forceful), or less turbulent, flow of vapor and gas within sterilization chamber 102 may be generated. In some embodiments, blower 106 may be configured to generate a stronger force to draw vapor and gas than, e.g., vacuum pump 110.

Vacuum pump 110 may be a vacuum pump having the capacity to draw gas from the interior (e.g., lower interior 103) of sterilization chamber 102, via vacuum conduit 112 and catalytic converter 115, and towards exhaust 116, thereby creating a vacuum within sterilization chamber 102 and/or a closed system containing sterilization chamber 102 and, e.g., blower 106. Vacuum pump 110 may be fluidly connected to exhaust 116 via, e.g., vacuum exhaust conduit 114. In some embodiments, exhausts from vacuum pump 110 and blower 106 may be separated instead of being combined into one.

In some embodiments, vacuum-type functions may also or alternately be performed by, e.g., blower 106, which may selectively circulate vapor and gas out of and into sterilization chamber 102 or out of sterilization chamber 102, through exhaust valve 120, and towards exhaust 116. Exhaust valve 120 may be selectively opened or closed so as to permit or prevent flow of gas or vapor from blower circulation conduit 118 towards exhaust 116 or towards inlet conduit 134 for reintroduction into sterilization chamber 102. Exhaust valve 120 may be manually controlled, or may be automatically controlled by, e.g., controller 140.

Catalytic converter 115, catalytic converter 121, or both may be, for example, any catalytic converters known in the art suitable for converting toxic gaseous or vaporized fluids circulated within sterilization system 100, e.g., during a sterilization cycle, to less toxic gases or vapors. For example, catalytic converters 115, 121 may be configured to convert VHP into water vapor, oxygen, and/or other non-toxic fluids.

A controller 140 is connected to one or more other components of sterilization system 100, such as sterilization chamber 102, temperature control jacket 104, blower 106, VHP injector 132, ambient air supply 117, dry air supply 127, auxiliary ambient air supply 130, vacuum pump 110, piston 150, catalytic converters 115, 121, condenser 147, distribution manifolds 107, 107b, conduits 112, 108, 118, 134, 135, valves 113, 119, 120, 124, 126, 144, and/or any other components of sterilization system 100. Some or all aspects of sterilization system 100 may be controllable by, e.g., controller 140. For example, controller 140 may be in communication with one or more valves 113, 119, 120, 124, 126, 144, and may be configured to control, adjust, and/or monitor the position of the one or more valves 113, 119, 120, 124, 126, 144. In addition or alternatively, the one or more valves 113, 119, 120, 124, 126, 144 may be manually operated.

Controller 140 may be, for example, an analog or digital controller configured to alter aspects of the environment of sterilization chamber 102 such as an internal temperature or pressure of sterilization chamber 102 and/or one or more of blower 106, vacuum pump 110, air supply 117, auxiliary ambient air supply 130, VHP injector 132, exhaust 116, one or more of valves 113, 119, 120, 124, 126, and 128, one or more of catalytic converters 115, 121, one or more of conduits 108, 112, 114, 116, 118, and 134, and any and/or other aspects of sterilization system 100. In some embodiments, sterilization system 100 may be controllable by multiple controllers 140. In other embodiments, sterilization system 100 may only have one controller 140. In some embodiments, controller 140 may be a digital controller, such as a programmable logic controller.

In some embodiments, controller 140 may be pre-programmed to execute one or more sterilization, aeration, drying, and/or cleaning cycles using sterilization system 100. In some embodiments, sterilization system 100 may be controllable by a controller having one or more human machine interface (“HMI”) elements, which may be configured to allow a user to input or alter desired parameters for a cycle, which may be executable by a controller on or operably coupled to sterilization system 100. Thus, in some embodiments, HMI elements may be used to program a customized cycle for execution by sterilization system 100. For example, in some embodiments, sterilization system 100 may be controllable by a controller connected to, e.g., a computer, tablet, or handheld device having a display. Such a display may include, for example, options to select or alter a desired temperature, pressure, time, amount of sterilant intake, amount of dry air, compressed air, or room air intake, etc., for one or more steps of a cycle.

The controller 140 may include one or more alarm system that stop a sterilization process or display an alert if an alarm condition is met. For example, if the sterilization chamber 102 fails to pressurize during a leak test, the controller 140 may stop the sterilization process. Other examples of alarm conditions that may trigger a stop of the sterilization process or displaying of an alert can include maximum or minimum temperature thresholds being exceeded, maximum or minimum pressure thresholds being exceeded, or a time of a step in the sterilization process taking longer than a pre-programmed threshold.

FIG. 1C depicts an expanded view of sterilization chamber 102. Sterilization chamber 102 may serve as an exemplary environment in which many aspects of the present disclosure may be applicable. It is to be understood, however, that sterilization chamber 102 is merely exemplary, and that aspects of the present disclosure may be applicable in many other environments.

Variations in temperature, pressure, humidity, and air/fluid flow may characterize different portions of sterilization chamber 102. For example, temperature control jacket 104 may be configured to control a temperature within sterilization chamber 102, but may have a more immediate effect on a periphery of sterilization chamber 102 than on a more central part of its interior. As has been described previously, temperature control jacket 104 may more immediately affect the temperature of products 105 (i.e., a load) closer to the periphery of sterilization chamber 102 than to the middle of sterilization chamber 102, causing products 105 closer to the periphery to be, e.g., warmer than products 105 in the middle.

As another example, distribution manifolds 107a, 107b and/or inlet 109 may have surface temperatures differing from an average interior temperature of sterilization chamber 102 (e.g., they may be cooler or warmer than an average interior temperature of sterilization chamber 102). Additionally, during operation of sterilization system 100, distribution manifolds 107a, 107b, inlet 109, a diaphragm (not pictured), and/or piston 150 may create localized areas of relatively higher pressure around themselves as they inject fluid into sterilization chamber 102 or create a pressure wave. This may cause greater condensation of fluids in areas closer to the source of the pressure wave as compared to areas farther away. For example, sterilant dispersed from one of the distribution manifolds 107a, 107b may be more likely to condense on products 105 located closer to distribution manifold 107a, 107b than products 105 that are farther away.

As has been described previously, products 105 may include one or more semi-permeable membranes through which vapor or gas may flow, such as a cover on packaging for a medical device or drug product. Semi-permeable membranes may, in some cases, not allow liquid to pass through them. In some embodiments, sterilization of areas on both sides of the semi-permeable membrane is desired.

FIG. 1B depicts in schematic form an exemplary sterilization system 500 for use in methods of the present disclosure. It is to be understood that sterilization system 500 is merely exemplary, and methods disclosed herein may be used in many other systems, environments, and/or parts thereof. For example, components, aspects, and/or structures of sterilization system 100 may be used in combination and/or as alternatives to components, aspects, and/or structures of sterilization system 500. Except for as described otherwise herein, components and aspects of sterilization system 500 may be structurally similar and/or function similarly to the similarly labeled components and aspects of sterilization system 100.

Still referring to FIG. 1B, sterilization system 500 may include a first distribution manifold 107a within an upper interior 101 of sterilization chamber 102. Sterilization system 500 may further include a second distribution manifold (not pictured) also within upper interior 101 of sterilization chamber 102. In some embodiments, sterilization system 500 does not include a second distribution manifold 107b. Sterilization system 500 may comprise a recirculation conduit 160, a recirculation blower 162, and a recirculation valve 164. Recirculation blower 162 may be fluidly connected to sterilization chamber 102 via recirculation conduit 160.

Recirculation conduit 160 may fluidly connect recirculation blower 162 to move fluids out of sterilization chamber 102 (e.g., at lower interior 103), through recirculation conduit 160, and back into sterilization chamber 102 (e.g., at upper interior 101). For example, recirculation conduit 160 may fluid connect to blower circulation conduit 118. A recirculation valve 164 may be located between recirculation blower 164 and sterilization chamber 102, and selectively closes or opens a connection between recirculation blower conduit 160 and sterilization chamber 102 (e.g., a connection including blower circulation conduit 118. Recirculation valve 164 may be positioned such that it is located between recirculation blower 162 and blower circulation conduit 118, as show in FIG. 1B. Alternatively, recirculation valve 164 may be positioned such that it is located between recirculation blower 162 and lower interior 103 of sterilization chamber 102.

Recirculation blower 162 may circulate air at a rate greater than 500 cubic feet per minute (cfm). In some embodiments, the rate of air circulation maintained by recirculation blower 162 may be less than 1000 cfm. If recirculation blower 162 is moving too much fluid, sterilant may be displaced near distribution manifold 107a, potentially negatively impacting a desired sterilant distribution. For example, if recirculation blower 162 is moving too much fluid, a variance in the distribution of sterilant within sterilization chamber 102 may be increased.

Recirculation blower 162 may be, for example, a blower having the capacity to forcibly draw vapor and gas from lower interior 103 of sterilization chamber 102 through recirculation conduit 160, and to reintroduce said vapor and gas back to upper interior 101 of sterilization chamber 102. In some embodiments, recirculation blower 162 may be external to sterilization chamber 102, as shown in FIG. 1B. In other embodiments, recirculation blower 162 may be disposed within sterilization chamber 102. In some embodiments, recirculation blower 162 may be configured to draw vapor and gas from lower interior 103 of sterilization chamber 102 and reintroduce said vapor and gas back to upper interior 101 with sufficient force to create a flow of vapor and gas from upper interior 101 to lower interior 103 of sterilization chamber 102. This flow may be termed a “turbulent flow.” In some embodiments, the force with which recirculation blower 162 may operate may be adjustable (via, for example, controller 140), such that a more turbulent (e.g., more forceful), or less turbulent, flow of vapor and gas within sterilization chamber 102 may be generated. In some embodiments, blower recirculation blower 162 may be configured to generate a stronger force to draw vapor and gas than, e.g., vacuum pump 110.

Sterilization system 500 may include an ambient air supply 117, a ambient air supply blower 122, and ambient air supply conduit 123. As shown in FIG. 1B, makeup air supply conduit 123 may extend from ambient air supply 117 to ambient air valve 124 with ambient air supply blower 122 disposed between ambient air supply 117 and ambient air valve 124.

In sterilization system 500, pressure change rates within sterilization chamber 102 may be controlled by controlling fluid flow rates through ambient air supply conduit 123 and inlet conduit 135, for example by controlling a relative flow rate between ambient air supply conduit 123 and inlet conduit 135. Controlling the relative flow rate between ambient air supply conduit 123 and inlet conduit 135 can be achieved by selecting an appropriately sized conduit such that a rate of pressure change by fluid introduced via ambient air supply conduit 123 is different than a rate of pressure change by fluid introduced via inlet conduit 135. For example, ambient air supply conduit 123 may have an inner diameter which is greater than the inner diameter of inlet conduit 135, such that a rate of pressure change by fluid introduced via ambient air supply conduit 123 is greater than a rate of pressure change by fluid introduce via inlet conduit 135.

Methods of the present disclosure may comprise one or more steps that include introducing gas (e.g., air) via ambient air supply conduit 123 and inlet conduit 135. For example, steps that include increasing a pressure of the sterilization chamber 102 to a pressure near atmospheric pressure, the majority of the gas (e.g., air) introduced to sterilization chamber 102 may be introduced via inlet conduit 135. The gas introduced to sterilization chamber 102 via inlet conduit 135 may increase a pressure of the sterilization chamber 102 to a pressure of approximately 850 mbar to approximately 1000 mbar, such as, for example, approximately 850 mbar to approximately 950 mbar, or approximately 900 mbar. After the gas is introduced via inlet conduit 135, the rest of the gas of the step may be introduced via ambient air supple conduit 123.

In some aspects, a ratio of an inner diameter of ambient air supply conduit 123 to an inner diameter of inlet conduit 135 may be selected to provide a desired difference in flowrates, and thereby pressure change rates between fluid introduced via ambient air supply conduit 123 relative to fluid introduced via inlet conduit 135. For example, a ratio of an inner diameter of ambient air supply conduit 123 to an inner diameter of inlet conduit 135 may be about 15:1 to about 5:1, such as, for example, about 11:1 to about 7:1, about 15:1, about 14:1, about 13:1 about 12:1, about 11:1, about 10:1, about 9.7:1, about 9.5:1, about 9.3:1, about 9:1, about 8.7:1, about 8.5:1, about 8.3:1, about 8:1, about 7.7:1, about 7.5:1, about 7.3:1, about 7:1, about 6.7:1, about 6.5:1, about 6.3:1, about 6:1, about 5.7:1, about 5.5:1, about 5.3:1, or about 5:1.

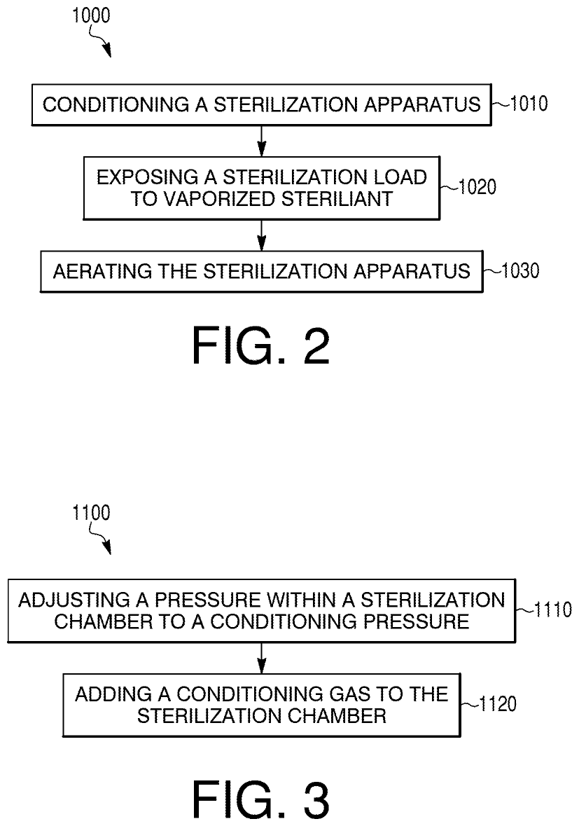

FIGS. 2, 3, 4A, 4B, and 5 depict flow diagrams of phases and steps in exemplary methods for sterilization according to the present disclosure. As will be recognized by one of ordinary skill in the art, some phases and/or steps may be omitted, combined, and/or performed out of order while remaining consistent with the present disclosure. In some embodiments, the phases and/or steps may be performed using, e.g., sterilization system 100, sterilization system 500, a variation of sterilization system 100, or a variation of sterilization system 500.

Additionally or alternatively, the phases and/or steps may be applicable in other environments in which manipulation of vaporized sterilant is desirable. It will be recognized that the customizable and controllable aspects of sterilization system 100 and/or sterilization system 500 may be used in order to carry out phases and steps described below. For example, in some embodiments, controller 140 may be employed to direct, adjust, or modify one or more of temperature, pressure, timings, etc. in a series of sterilization steps, setpoints, and phases performable by sterilization system 100 and/or sterilization system 500. Additionally, although the phases and steps described below are recited in relation to sterilization system 100 and/or sterilization system 500, one of ordinary skill in the art will understand that these phases and steps may be performed by another sterilization system, or another system having the capacity to carry out the steps.

FIG. 2 depicts a flow diagram of a series of steps in an exemplary sterilization method 1000. Sterilization method 1000 may include conditioning a sterilization apparatus (step 1100). Sterilization method 1000 may include exposing a sterilization load to vaporized sterilant (step 1200). Sterilization 1000 may include aerating the sterilization apparatus (step 1300). The sterilization method 1000 may be performed with a sterilization system, such as, for example, sterilization system 100.

Prior to performance of the steps of sterilization method 1000, a sterilization load, such as products 105, may be placed within a sterilization chamber, such as sterilization chamber 102, of a sterilization system, such as sterilization system 100 and/or sterilization system 500. A closed-system sterilization environment—including, for example, sterilization chamber 102, blower exit conduit 108, blower 106, blower circulation conduit 118, inlet conduit 134, condenser 147, and any elements connecting these components—may then be sealed. In some cases, a leak test may be performed on the closed-system sterilization environment. The leak test may include, for example, creating a vacuum through the closed system. The vacuum may be created by, e.g., expelling gas and vapor from the closed system using vacuum pump 110. During the leak test, blower 106 may be in operation, so as to circulate any remaining air through the closed system and create a homogenous environment. The leak test may be performed in this manner in part to verify that a suitable vacuum may be held within the closed system.

For example, a leak test may include adjusting a pressure of sterilization chamber 102 to a leak test pressure for a stabilization time, holding the sterilization chamber 102 at the leak test pressure for a leak test duration, and measuring a total leak. If the pressure of the sterilization chamber 102 decreases by more than an acceptable leak threshold, the sterilization method 1000 may be aborted and the sterilization chamber 102 may be resealed. An acceptable total leak threshold may be about 20 millibars, about 18 millibars, about 15 millibars, about 13 millibars, about 10 millibars, about 8 millibars, or about 5 millibars. The leak test pressure may be at least 450 millibars, at least 500 millibars, at least 550 millibars, at least 600 millibars, at least 650 millibars, at least 700 millibars, at least 750 millibars, about 450 millibars, about 500 millibars, about 550 millibars, about 600 millibars, about 650 millibars, about 700 millibars, or about 750 millibars.

In some embodiments, the sterilization system (e.g., sterilization system 100 or sterilization system 500) may be conditioned. FIG. 3 depicts a flow diagram with steps in an exemplary method 1100 of conditioning a sterilization apparatus. Conditioning a sterilization apparatus may include adjusting a pressure within a sterilization chamber (e.g., sterilization chamber 102) to a conditioning pressure (step 1110). Suitable conditioning pressures may be at least 450 millibars, at least 500 millibars, at least 550 millibars, at least 600 millibars, at least 650 millibars, at least 700 millibars, at least 750 millibars, about 450 millibars, about 500 millibars, about 550 millibars, about 600 millibars, about 650 millibars, about 700 millibars, or about 750 millibars. Conditioning a sterilization apparatus may further include adding a conditioning gas to the sterilization chamber (step 1110). The conditioning gas may be a dry gas, such as a gas containing nitrogen (e.g., commercially available supplies of only nitrogen or primarily nitrogen), or air having a dew point of, for example, −10 °C or less. The use of a dry gas may be selected to allow for ample air exchange and reduction in humidity within a sterilization chamber (e.g., sterilization chamber 102), to further avoid unnecessary condensation. After the conditioning gas is added, the pressure of the sterilization chamber may be at least 550 millibars, at least 600 millibars, at least 650 millibars, at least 700 millibars, at least 750 millibars, at least 800 millibars, at least 850 millibars, at least 900 millibars, at least 950 millibars, about 450 millibars, about 500 millibars, about 550 millibars, about 600 millibars, about 650 millibars, about 700 millibars, about 750 millibars, about 800 millibars, or about 850 millibars. Together, steps 1110 and 1120 of conditioning a sterilization apparatus may be referred to as a conditioning pulse. Conditioning a sterilization apparatus may include multiple conditioning pulses, such as for example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 conditioning pulses.

In addition or alternatively, the method 1100 of conditioning may include, for example, increasing the temperature of the closed system to temperatures intended to be maintained during a sterilization phase (e.g., between about 25 °C and about 50 °C). In some embodiments, conditioning may be performed for longer than is performed in standard chemical sterilization procedures, which may allow more time for any temperature differences between the environment in the closed system (including, e.g., areas of a sterilization chamber such as sterilization chamber 102) to decrease. Alternatively or additionally, conditioning may include, for example, pairing a temperature of a temperature control jacket (e.g., temperature control jacket 104) with a temperature of an inlet, such as first distribution manifold 107a, second distribution manifold 107b, and/or inlet 109, prior to or for the duration of method 1100. By “pairing” the temperature of the temperature control jacket with the temperature of an inlet, it is meant that the temperature control jacket is programmed to maintain the same or a similar temperature as a surface temperature of an inlet inside the sterilization chamber. This may be advantageous because inlets (e.g., distribution manifolds 107a, 107b and/or inlet 109) may generally be cooler than other parts of the sterilization chamber, due to the temperature of, e.g., sterilant, compressed air, or other fluid traveling through them. Additionally, as the temperature control jacket 104 warms the sterilization chamber 102, the periphery of the chamber (e.g., a portion of the sterilization chamber 102 closest to the temperature control jacket) may be warmer than the middle of the sterilization chamber (e.g., farthest from the temperature control jacket). Thus, pairing the temperature control jacket with the temperature of an inlet may reduce temperature differences across the sterilization chamber. In some embodiments, the temperature control jacket may be paired by, e.g., setting the temperature control jacket to a known temperature of an inlet during a sterilization cycle. In some embodiments, the temperature control jacket may be paired by, e.g., experimentally determining a temperature of an inlet during one or more sterilization cycles and setting the temperature control jacket to that temperature. In other embodiments, a digital thermometer may be disposed in contact with or near an inlet (e.g., a distribution manifold 107a, 107b, or inlet 109), which may relay temperature information to a controller (e.g., controller 140). For example, one or more thermometers or temperature sensors (not shown) may transmit temperature information to the controller 140 when prompted, periodically, continuously, or dynamically (e.g., periodically during the sterilization cycle, based on monitored conditions of the chamber).

The controller 140 may, in turn, set the temperature control jacket 104 to the temperature sensed at or near the inlet (e.g., distribution manifolds 107a, 107b, or inlet 109). As surface temperatures of distribution manifolds 107a, 107b, and inlet 109 may in general be cooler than an average internal temperature of sterilization chamber 102, pairing the temperature of the temperature control jacket 104 with the temperatures of an inlet (e.g., distribution manifolds 107a, 107b, or inlet 109) may rectify some differences in temperature existing throughout the sterilization chamber, which may, in turn, aid in distribution of sterilant throughout the sterilization chamber.

It is also contemplated that, in some embodiments, maintaining a temperature differential between the sterilization load and the surrounding closed system, creating “cold spots” via may have advantages. For example, controlled condensation of vaporized sterilizing chemical (e.g., VHP) on “cold spots” of the load may concentrate the sterilizing chemical on the load and lead to more efficient diffusion of the chemical into the load, thus decreasing the overall amount of sterilizing chemical needed in the sterilization chamber 102 to achieve effective sterilization. In such embodiments, it may be advantageous to reduce a time for preconditioning, or to eliminate preconditioning entirely.

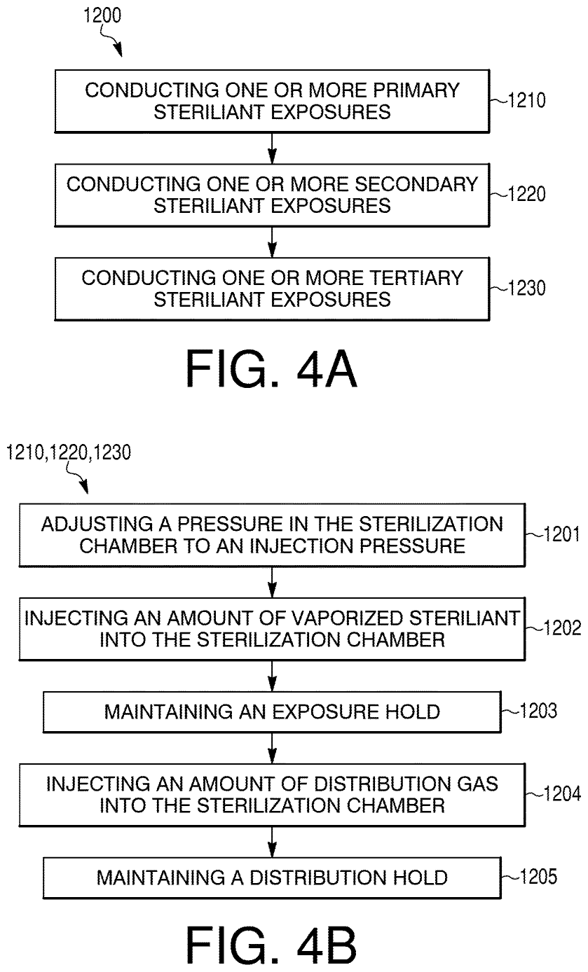

As described herein, the sterilization method 1000 may include exposing a sterilization load to vaporized sterilant. FIG. 4A depicts a flow diagram with steps in an exemplary method 1200 of exposing a sterilization load to vaporized sterilant. Exposing a sterilization load to vaporized sterilant may include conducting one or more primary sterilant exposures (step 1210), conducting one or more second sterilant exposures (step 1220), and/or conducting one or more tertiary sterilant exposures (step 1230).

FIG. 4B depicts a flow diagram with steps in an exemplary method (1210, 1220, 1230) of a sterilant exposure, such as, for example, a primary sterilant exposure, a second sterilant exposure, or a tertiary sterilant exposure. The method 1210, 1220, 1230 of the sterilant exposure may include adjusting a pressure in the sterilization chamber to an injection pressure (step 1201), injecting an amount of vaporized sterilant into the sterilization chamber (step 1202), maintaining an exposure hold (step 1203), injecting an amount of distribution gas into the sterilization chamber (step 1204), and maintaining a distribution hold (step 1205).

The injection pressure may be, for example, about 400 millibars to about 1000 millibars, about 450 millibars to about 1000 millibars, about 400 millibars to about 900 millibars, about 450 millibars to about 850 millibars, about 400 millibars to about 800 millibars, about 450 millibars to about 800 millibars, about 450 millibars to about 750 millibars. For example, the injection pressure may be about 400 millibars, about 450 millibars, about 500 millibars, about 550 millibars, or about 600 millibars. The injection pressure may promote a higher concentration of sterilant on the sterilization load, and increasing exposure of the sterilization load to the sterilant.

Injecting an amount of vaporized sterilant into the sterilization chamber may include injecting the vapor produced by vaporizing a solution including the sterilant. In some aspects, the solution including the sterilant may be an aqueous hydrogen peroxide solution, having a concentration of, for example, between about 5% and about 75% hydrogen peroxide by weight. In some embodiments, the vaporized chemical may be a vaporized aqueous hydrogen peroxide solution having a concentration of, for example, between about 10% and about 65% hydrogen peroxide by weight, between about 15% and about 60% hydrogen peroxide by weight, between about 30% and about 60% hydrogen peroxide by weight, between about 30% and about 60% hydrogen peroxide by weight, or between about 45% and about 60% hydrogen peroxide by weight. In some embodiments, the vaporized chemical may be a vaporized aqueous hydrogen peroxide having a concentration of about 35% hydrogen peroxide (and 65% water) by weight. In further embodiments, the vaporized chemical may be a vaporized aqueous hydrogen peroxide having a concentration of about 59% hydrogen peroxide (and 41% water) by weight.

The amount of vaporized chemical injected into the sterilization chamber may be characterized by the mass of solution including the sterilant that was used to generate the vapor. The injected amount of vaporized sterilant may be about 50 g to about 350 g, about 50 g to about 300 g, about 50 g to about 250 g, about 50 g to about 200 g, about 100 g to about 300 g, about 100 g to about 250 g, about 100 g to about 200 g, about 100 g to about 175 g, or about 125 g to about 200 g. For example, the injected amount of vaporized sterilant may be about 50 g, about 75 g, about 90 g, about 100 g, about 110 g, about 125 g, about 130 g, about 140 g, about 150 g, about 160 g, about 175 g, about 180 g, about 190 g, or about 200 g.

In some aspects, amount of injected sterilant may be quantified based on the volume or amount of load to be sterilized inside sterilization chamber 102. For example, if a number of drug products, such as pre-filled syringes, are to be sterilized in sterilization chamber 102, an injected amount of vaporized sterilant may be about 0.01 to about 0.25, about 0.01 to about 0.20, about 0.01 to about 0.15, about 0.05 to about 0.25, about 0.05 to about 0.20, about 0.05 to about 0.25, about 0.05 to about 0.15, or about 0.015 to about 0.25 grams per unit drug product. In other aspects, an amount of injected sterilant may be quantified based on the volume of the sterilization environment, such as the interior of sterilization chamber 102. For example, an amount of injected sterilant may be about 0.25 grams, about 0.50 grams, about 0.75 grams, about 1.0 gram, about 1.2 grams, about 1.4 grams, about 1.5 grams, about 1.6 grams, about 1.8 grams, about 2.0 grams, about 2.2 grams, about 2.5 grams, or about 3 grams per cubic foot of volume in a sterilization chamber.

The total amount of vaporized sterilant injected into the sterilization chambers across all exposures of vaporized sterilant to the sterilization load may be about 600 grams to about 1000 grams, about 600 grams to about 950 grams, about 650 grams to about 1000 grams, about 650 grams to about 950 grams, about 650 grams to about 900 grams, about 700 grams to about 1000 grams, about 700 grams to about 950 grams, about 700 grams to about 900 grams, about 750 grams to about 1000 grams, about 750 grams to about 950 grams, about 750 grams to about 900 grams, about 800 grams to about 900 grams, about 600 grams, about 650 grams, about 700 grams, about 725 grams, about 750 grams, about 775 grams, about 800 grams, about 825 grams, about 850 grams, about 875 grams, about 900 grams, or about 925 grams.

Referring still to FIG. 4B, after the amount of vaporized sterilant is injected into the sterilization chamber at step 1202, an exposure hold may be maintained at step 1203. The time for which an exposure hold is maintained (an exposure hold time) may be selected so as to allow the vaporized sterilant adequate time to contact the load, without allowing excess condensation of the vaporized sterilant. In some embodiments, the exposure hold time may be at least at least 30 seconds, at least 45 seconds, at least 60 seconds, at least 75 seconds, at least 120 seconds, at least 150 seconds, at least 190 seconds, about 45 seconds, about 60 seconds, about 75 seconds, or about 90 seconds.

After the exposure hold at step 1203, a distribution gas may be injected into the sterilization chamber at step 1204. Addition of distribution gas may increase the pressure in the sterilization chamber to a transition pressure. The gas may be any suitable gas that can lessen the vacuum in sterilization chamber 102. In some aspects, the distribution gas may be a dry gas, such as a gas containing nitrogen (e.g., commercially available supplies of only nitrogen or primarily nitrogen), or air having a dew point of, for example, −10 °C or less. The use of a dry gas may be selected to allow for ample air exchange and reduction in humidity within a sterilization chamber, to further avoid excess condensation. In some embodiments, distribution gas may be injected from ambient air supply 117, dry air supply 127, and/or auxiliary ambient air supply 130. The amount of distribution gas injected may be an amount sufficient to achieve a transition pressure of about 750 millibars to about 1100 millibars, about 750 millibars to about 1100 millibars, about 750 millibars to about 1000 millibars, about 800 millibars to about 1100 millibars, about 800 millibars to about 1000 millibars, about 850 millibars to about 1000 millibars, about 850 millibars to about 950 millibars, about 900 millibars to about 1000 millibars, about 800 millibars, about 850 millibars, about 900 millibars, about 910 millibars, about 920 millibars, about 930 millibars, about 940 millibars, about 950 millibars, about 960 millibars, about 970 millibars, about 980 millibars, or about 1000 millibars, within the sterilization chamber.

In instances where a sterilization load includes semi-permeable membranes through which it is desired that sterilant pass, the increase in pressure caused by addition of the distribution gas may serve to help migrate the sterilant through such semi-permeable membranes.

After an amount of distribution gas is injected into the sterilization chamber at step 1204, a distribution hold may be maintained at step 1205. The amount of time that the distribution hold is maintained for may be referred to as the distribution hold time. During the distribution hold, the pressure achieved during from the injection distribution gas may be maintained for at least 30 seconds, at least 1 minute, at least 2 minutes, at least 3 minutes, at least 4 minutes, at least 5 minutes, at least 6 minutes, at least 7 minutes, at least 8 minutes, about 30 seconds, about 1 minute, about 2 minutes, about 3 minutes, about 4 minutes, about 5 minutes, about 6 minutes, about 7 minutes, about 8 minutes, about 9 minutes, or about 10 minutes.

A single iteration of a sterilant exposure (e.g., method 1210, 1220, 1230) may be referred to as a sterilization pulse. The one or more primary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses. The one or more secondary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses. The one or more tertiary sterilant exposures may include about 2 to about 15 sterilization pulses, about 2 to about 12 sterilization pulses, about 2 to 10 sterilization pulses, about 2 to about 8 sterilization pulses, about 2 to about 6 sterilization pulses, about 2 to about 5 sterilization pulses, about 2 to about 5 sterilization pulses, such as, for example, 2 pulses, 3 pulses, 4 pulses, 5 pulses, 6 pulses, 7 pulses, or 8 pulses.

Parameters of a sterilant exposure may include an amount of sterilant added, a duration of the sterilant injection, an exposure hold time, a transition pressure, and a distribution hold time. Each primary sterilant exposure may have the same parameters as each other primary sterilant exposure. Each secondary sterilant exposure may have the same parameters as each other secondary sterilant exposure. Each tertiary sterilant exposure have the same parameters as each other tertiary sterilant exposure. A primary sterilant exposure may have at least one parameter that is different than a secondary sterilant exposure and/or at least one parameter that is different than a tertiary sterilant exposure. In addition or alternatively, a secondary sterilant exposure may have at least one parameter that is different than a tertiary sterilant exposure.

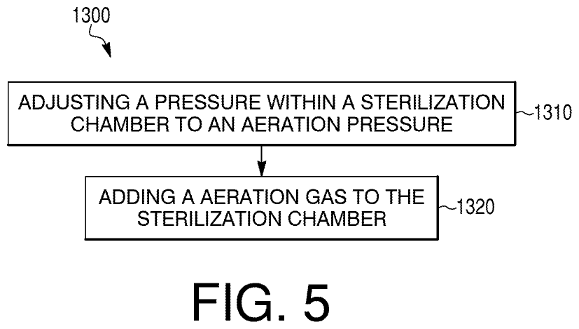

FIG. 5 depicts a flow diagram with steps in an exemplary method 1300 of aerating the sterilization apparatus. Aerating the sterilization apparatus may include adjusting a pressure within a sterilization chamber to an aeration pressure (step 1310). Aerating the sterilization apparatus may also include adding an aeration gas to the sterilization chamber (step 1320).

One iteration of adjusting a pressure within the sterilization chamber to an aeration pressure (step 1310) and adding an aeration gas to the sterilization chamber (step 1320) may be referred to as an aeration pulse. Aerating the sterilization apparatus may include multiple aeration pulses. For example, aerating the sterilization apparatus may include at least 1 aeration pulse, at least 2 aeration pulses, at least 3 aeration pulses, at least 4 aeration pulses, at least 5 aeration pulses, at least 6 aeration pulses, at least 7 aeration pulses, at least 8 aeration pulses, at least 9 aeration pulses, at least 10 aeration pulses, 1 aeration pulse, 2 aeration pulses, 3 aeration pulses, 4 aeration pulses, 5 aeration pulses, 6 aeration pulses, 7 aeration pulses, 8 aeration pulses, 9 aeration pulses, or 10 aeration pulses.

The aeration pressure may be about 400 millibars to about 1000 millibars, about 400 millibars to about 900 millibars, about 400 millibars to about 600 millibars, about 450 millibars to about 900 millibars, about 450 millibars to about 600 millibars, about 450 millibars to about 550 millibars, at least 400 millibars, at least 450 millibars, at least 475 millibars, at least 500 millibars, at least 525 millibars, at least 550 millibars, at least 575 millibars, at least 600 millibars, about 400 millibars, about 450 millibars, about 475 millibars, about 500 millibars, about 525 millibars, about 550 millibars, about 575 millibars, or about 600 millibars.

The aeration gas may comprise a dry gas, such as a gas containing nitrogen (e.g., commercially available supplies of only nitrogen or primarily nitrogen), or air having a dew point of, for example, −10 °C or less. Adding an aeration gas to the sterilization chamber may raise the pressure of the sterilization chamber. In some aspects, adding the aeration gas to the sterilization chamber may raise the pressure of the sterilization chamber by about 50 millibar to about 150 millibar to an exhaust pressure. For example, after the aeration gas is added to the sterilization chamber at step 1320, the exhaust pressure of the sterilization chamber may be 500 millibars to about 1100 millibars, about 500 millibars to about 1000 millibars, about 500 millibars to about 700 millibars, about 550 millibars to about 1000 millibars, about 550 millibars to about 700 millibars, about 550 millibars to about 650 millibars, at least 500 millibars, at least 550 millibars, at least 575 millibars, at least 600 millibars, at least 625 millibars, at least 650 millibars, at least 675 millibars, at least 800 millibars, about 500 millibars, about 550 millibars, about 575 millibars, about 600 millibars, about 625 millibars, about 650 millibars, about 675 millibars, or about 700 millibars.

After the pressure in the sterilization chamber is raised by the addition of the aeration gas a step 1320, the pressure in the sterilization chamber may need to be adjusted to an aeration pressure (step 1310) for a subsequent aeration pulse. The pressure may be lowered by venting the sterilization apparatus, and exhausting gases within the sterilization chamber. During this step, blower 106 may be turned on while recirculation valve 119 is closed and exhaust valve 120 is opened, such that blower 106 pulls fluid from within sterilization chamber 102 and expels it through exhaust 116 via catalytic converter 121. Because blower exit conduit 108 is connected to sterilization chamber 102 at lower interior 103 of sterilization chamber 102, denser fluids that have settled to lower interior 103 (such as sterilizing chemicals) may be removed by this step. Air (e.g., from ambient air supply 117, dry air supply 127, and/or auxiliary ambient air supply 130) may be concurrently allowed to vent into sterilization chamber 102.

The amount of time required to transition the pressure in the sterilization chamber from the pressure resulting from the addition of the aeration gas to the aeration pressure (step 1320) of the subsequent aeration pulse may be referred to as an exhaust time. An exhaust time may be instantaneous (i.e., an exhaust time of 0 minutes), or the decrease in pressure may be more gradual. For example, the exhaust time of an aeration pulse may be at least 1 minute, at least 3 minutes, at least 5 minutes, at least 7 minutes, at least 10 minutes, at least 12 minutes, at least 15 minutes, at least 18 minutes, at least 20 minutes, at least 22 minutes, at least 25 minutes, at least 28 minutes, at least 30 minutes, at least 32 minutes, at least 35 minutes, at least 37 minutes, at least 40 minutes, at least 45 minutes, at least 50 minutes, at least 55 minutes, at least 60 minutes, about 1 minute, about 3 minutes, about 5 minutes, about 7 minutes, about 10 minutes, about 12 minutes, about 15 minutes, about 18 minutes, about 20 minutes, about 22 minutes, about 25 minutes, about 28 minutes, about 30 minutes, about 32 minutes, about 35 minutes, about 37 minutes, about 40 minutes, about 45 minutes, about 50 minutes, about 55 minutes, or about 60 minutes.

Parameters of an aeration pulse may include a sterilization pressure, an amount of aeration gas added, a pressure achieved by the addition of the aeration gas, and/or an exhaust time. Each aeration pulse may have the same parameters as each other aeration pulse. An aeration pulse may have different parameters than a previous or subsequent aeration pulse. An aeration pulse may have the same parameters as a previous and/or subsequent aeration pulse.

In some methods, the pressure of the sterilization chamber 102 may be adjusted (step 1310) while the aeration gas is added to the sterilization chamber 102 (step 1320). For example, a vacuum may be drawn in the sterilization chamber 102 while also injecting aeration gas into sterilization chamber 102 near upper interior 101 of sterilization chamber 102, such as via first distribution manifold 107a or inlet 109 and/or near lower interior 103 of sterilization chamber 102 via second distribution manifold 107b. The aeration gas may be injected from, for example, ambient air supply 117, dry air supply 127, and/or auxiliary ambient air supply 130. While dry gas is being injected into sterilization chamber 102, a vacuum may be pulled by, e.g., vacuum pump 110 via vacuum conduit 112, catalytic converter 115, and vacuum exhaust conduit 114. The vacuum may be pulled at a greater rate than the rate of injection of dry gas, such that a vacuum level is gradually achieved. Injection of the dry gas near upper interior 101 of sterilization chamber 102 while achieving a desired vacuum level reduces condensation of VHP and water vapor at upper interior 101 of the sterilization chamber 102, and promotes the movement of denser molecules in sterilization chamber 102 towards the lower interior (e.g., lower interior 103) of sterilization chamber 102, and to some extent out of sterilization system 100 through vacuum exhaust conduit 114. The addition of more dry gas may continue to force sterilization chemicals to settle to the lower interior 101 of sterilization chamber 102, thus moving them away from the sterilization load and positioning them for removal via vacuum conduit 112 or blower exit conduit 108.

In some methods, before performing a sterilization or aeration pulses, it may be desirable to achieve and/or maintain a localized climate suitable for sterilization or aeration. Maintaining a localized climate may generally refer to ensuring that one or more areas within a sterilization system (e.g., sterilization system 100 or sterilization system 500) exhibit conditions (e.g., temperature, pressure, water vapor concentration, sterilant concentration, etc.) suitable for upcoming steps in a sterilization method. Maintenance of consistent or targeted localized climates may aid in robust sterilization and aeration efficacy. Localized climates may attract or repel sterilant to one or more specific localities within a system. If controlled, such localized climates may assist in achieving a desired level of sterilization. For example, inlets and/or carts supporting the sterilization load may be heated to prevent condensation of sterilant. The sterilization load may be kept at a temperature cooler than the carts to attract VHP or promote adhesion and condensation. Larger loads may also be used to reduce peak load temperature.

Furthermore, maintaining localized climates may aid in distributing sterilant to, or removing sterilant from, more difficult localities within a sterilization system, thus reducing the amount of “overkill” needed to sterilize or aerate those localities. These localities may include surfaces and interior regions of packaging materials. Attracting sterilant to a locality that has previously proven to struggle passing sterilization standards (e.g., biological indicator metrics) may normalize sterilization of that zone without the need to add more sterilant. Similarly, removing sterilant from a locality that has previously been difficult to aerate may reduce overall aeration and drying times in the methods described herein.

Maintaining localized climates may be accomplished by changing a temperature of one or more localities to reduce the temperature of localities to which sterilant should move. For example, the periphery of a sterilization chamber may be warmer than the middle of the chamber, due to heating elements located around the periphery of the chamber (e.g., temperature control jacket 104). Maintaining a localized climate for sterilization at the periphery of a sterilization chamber may thus include decreasing the temperature of the periphery of the sterilization chamber by, e.g., decreasing a target temperature of a heating element (e.g., temperature control jacket 104). In some examples, prior to or during an aeration pulse, a temperature in the sterilization chamber or sterilization system may be increased. This may increase movement of sterilant out of the sterilization chamber 102.

In some embodiments, any or all of the above-described steps and phases may be executed automatically by a sterilization system (e.g., sterilization system 100 or sterilization system 500) as directed by, e.g., controller 140, which may be programmed or otherwise configured in advance by e.g., a user. The methods of sterilization disclosed herein may be qualified as “limited overkill” sterilization methods, in that they may ensure sterilization of a load of, e.g., PFS while minimizing impact of the sterilization method on the product.

During operation of a sterilization method (e.g., method 1000), conditions experienced by the sterilization load may be monitored to confirm that an effective sterilization occurred without damaging the drug substance within the sterilization load. Conditions that may be monitored include total amount of sterilant injected into the sterilization chamber 102, a maximum temperature of the sterilization load, a minimum pressure of the sterilization load, and/or a total duration of the exposure to vaporized sterilant.

Chemical indicators may be placed throughout the sterilization load to ensure exposure of vaporized sterilant to all portions of the sterilization load. In addition or alternatively, biological indicators may be placed throughout the sterilization load to ensure effectiveness of the vaporized sterilant to all portions of the sterilization load. Chemical indicators may be configured to change color when exposed to a threshold amount of a specific sterilant. Biological indicators may include one or more microorganisms, including, for example, microorganisms with a tolerance for a specific sterilant. In the context of sterilization methods utilizing vaporized hydrogen peroxide, biological indicators may include Geobacillus stearothermophilus cultures with spores that have hydrogen peroxide resistance. After their use in a sterilization method, the biological indicators may be tested in a lab for growth to confirm effectiveness of the sterilization method.

In some examples, a sterilization method may be considered to have effectively sterilized the sterilization load without adversely affecting drug product in the load when one or more of the following conditions are met: the maximum temperature of the sterilization load did not exceed 35 °C, the minimum pressure of the sterilization chamber did not go below 480 millibars, the total duration of exposure of the sterilization load to the vaporized sterilant was at least 3 hours, all chemical indicators change color, and/or all biological indicators show negative growth.

Multiple sterilization and aeration phases have been described herein. It is to be understood that characteristics, methods, or steps of any one sterilization phase may be applied to any other sterilization method described herein. Likewise, characteristics, methods, or steps of any one aeration phase described herein may be applied to any other aeration phase or method.

### EXAMPLES

The following examples are intended to illustrate the present disclosure without being limiting in nature. It is understood that the present disclosure encompasses additional aspects and embodiments consistent with the foregoing description and following examples.

### Example 1

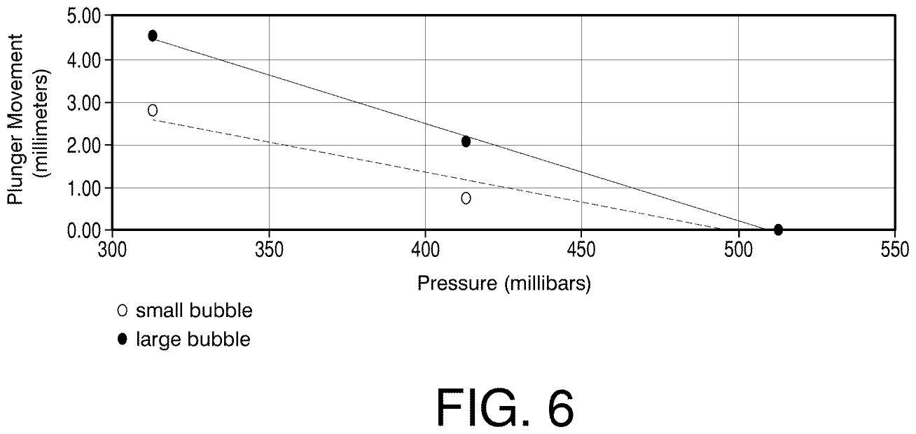

During development of the prefilled syringes, it was determined that the plunger of the prefilled syringe could move up to 2.4 mm before contacting the liquid medicament in the syringe. Syringes with two different air bubble sizes: small (approximately 2 mm diameter) and large (approximately 4 mm diameter) were analyzed at pressures of 313 millibars, 413 millibars, and 513 millibars. The movement of the plunger at each pressure was plotted as a function of the pressure. Linear regression was used to determine a line of best fit for each bubble size, correlating a relationship between plunger movement and pressure. The resulting plot and lines of best fit are shown in FIG. 6. The line of best fit for the small bubble was y=−0.014×+6.938, where x is the pressure in millibars and y is the movement of the plunger in millimeters. The line of best fit for the large bubble was y=−0.0228×+11.602, where x is the pressure in millibars and y is the movement of the plunger in millimeters.

The lines of best fit were used to extrapolate plunger movement at 480 millibars. As shown in FIG. 6, this plunger movement at 480 millibars is about 0.22-0.66 mm, depending on the bubble size. Based on this analysis, a minimum sterilization pressure of 480 millibars was determined to have no impact on the sterile boundary between plunger and liquid medicament.

### Example 2

A sterilization method (Sterilization Method I) was performed on two different sterilization loads, Sterilization Load A and Sterilization Load B, according to the phases and parameters shown in Table 1. Sterilization Method I included two primary sterilant exposures, three secondary sterilant exposures, seven tertiary sterilant exposures, and seven aeration pulses. Each primary sterilant exposure had the same parameters as the other primary sterilant exposure, each secondary sterilant exposure had the same parameters as the other secondary sterilant exposures, and each tertiary sterilant exposure had the same parameters as the other tertiary sterilant exposures. Sterilization Load A included eight carts, with each cart including 1,368 prefilled syringes. Sterilization Load B included eight carts, with each cart including 76 prefilled syringes. Each prefilled syringe of Sterilization Load A and Sterilization Load B included about 0.19 grams of liquid medicament (i.e., about 0.18 mL). Each sterilization load also included 144 biological indicators placed inside blister packages and distributed throughout the sterilization load.

TABLE 1

| | | Value for | | Value for | |
|---|---|---|---|---|---|
| | | Sterilization | | Sterilization | |
| Phase | Parameter | Method I | | Method II | |
| Leak Test | Vacuum Level | 500 | mbar | 500 | mbar |
| Leak Test | Stabilization Time | 2 | minutes | 2 | minutes |
| Leak Test | Leak Test Time | 5 | minutes | 5 | minutes |
| Leak Test | Acceptable Total Leak | 13 | mbar | 13 | mbar |
| Conditioning | Pulse Quantity | 12 | | 12 | |
| Conditioning | Conditioning Pressure | 500 | mbar | 500 | mbar |
| Conditioning | Conditioning Pressure Break Point | 700 | mbar | 700 | mbar |
| Conditioning | Jacket and Doors Temperature | 28 | °C | 28 | °C |
| Conditioning | Vaporizer Temperature | 110 | °C | 110 | °C |
| Primary Sterilant Exposure | Number of Sterilization Pulses | 2 | | 2 | |
| Primary Sterilant Exposure | Injection Pressure | 500 | mbar | 500 | mbar |
| Primary Sterilant Exposure | H₂O₂ (59 wt. % solution) Injection Amount | 150 | g | 150 | g |
| Primary Sterilant Exposure | Injection Cycle Duration | 7000 | msec | 7000 | msec |
| Primary Sterilant Exposure | Exposure Hold Time | 1 | minute | 1 | minute |
| Primary Sterilant Exposure | Transition Pressure | 940 | mbar | 940 | mbar |
| Primary Sterilant Exposure | Distribution Hold Time | 4 | minutes | 4 | minutes |
| Secondary Sterilant Exposure | Number of Sterilization Pulses | 3 | | 2 | |
| Secondary Sterilant Exposure | Injection Pressure | 500 | mbar | 500 | mbar |
| Secondary Sterilant Exposure | H₂O₂ (59 wt. % solution) Injection Amount | 75 | g | 75 | g |
| Secondary Sterilant Exposure | Injection Cycle Duration | 7000 | msec | 7000 | msec |
| Secondary Sterilant Exposure | Exposure Hold Time | 1 | minute | 1 | minute |
| Secondary Sterilant Exposure | Transition Pressure | 940 | mbar | 940 | mbar |
| Secondary Sterilant Exposure | Distribution Hold Time | 4 | minutes | 4 | minutes |
| Tertiary Sterilant Exposure | Number of Sterilization Pulses | 7 | | 6 | |
| Tertiary Sterilant Exposure | Injection Pressure | 600 | mbar | 600 | mbar |
| Tertiary Sterilant Exposure | H₂O₂ (59 wt. % solution) Injection Amount | 50 | g | 50 | g |
| Tertiary Sterilant Exposure | Injection Cycle Duration | 7000 | msec | 7000 | msec |
| Tertiary Sterilant Exposure | Exposure Hold Time | 1 | minute | 1 | minute |
| Tertiary Sterilant Exposure | Transition Pressure | 900 | mbar | 900 | mbar |
| Tertiary Sterilant Exposure | Distribution Hold Time | 10 | minutes | 10 | minutes |
| First Aeration | Vacuum Level | 500 | mbar | 500 | mbar |
| First Aeration | Exhaust Pressure | 940 | mbar | 940 | mbar |
| First Aeration | Exhaust Time | 35 | minutes | 35 | minutes |
| Second Aeration | Vacuum Level | 500 | mbar | 500 | mbar |
| Second Aeration | Exhaust Pressure | 700 | mbar | 700 | mbar |
| Second Aeration | Exhaust Time | 0 | minutes | 0 | minutes |
| Third Aeration | Vacuum Level | 500 | mbar | 500 | mbar |
| Third Aeration | Exhaust Pressure | 940 | mbar | 940 | mbar |
| Third Aeration | Exhaust Time | 15 | minutes | 15 | minutes |
| Fourth Aeration | Vacuum Level | 500 | mbar | 500 | mbar |
| Fourth Aeration | Exhaust Pressure | 940 | mbar | 940 | mbar |
| Fourth Aeration | Exhaust Time | 15 | minutes | 15 | minutes |
| Fifth Aeration | Vacuum Level | 500 | mbar | 500 | mbar |
| Fifth Aeration | Exhaust Pressure | 940 | mbar | 940 | mbar |
| Fifth Aeration | Exhaust Time | 15 | minutes | 15 | minutes |
| Sixth Aeration | Vacuum Level | 500 | mbar | 500 | mbar |
| Sixth Aeration | Exhaust Pressure | 600 | mbar | 600 | mbar |
| Sixth Aeration | Exhaust Time | 0 | minutes | 0 | minutes |
| Seventh Aeration | Vacuum Level | 900 | mbar | 900 | mbar |
| Seventh Aeration | Exhaust Pressure | 940 | mbar | 940 | mbar |
| Seventh Aeration | Exhaust Time | 15 | minutes | 15 | minutes |

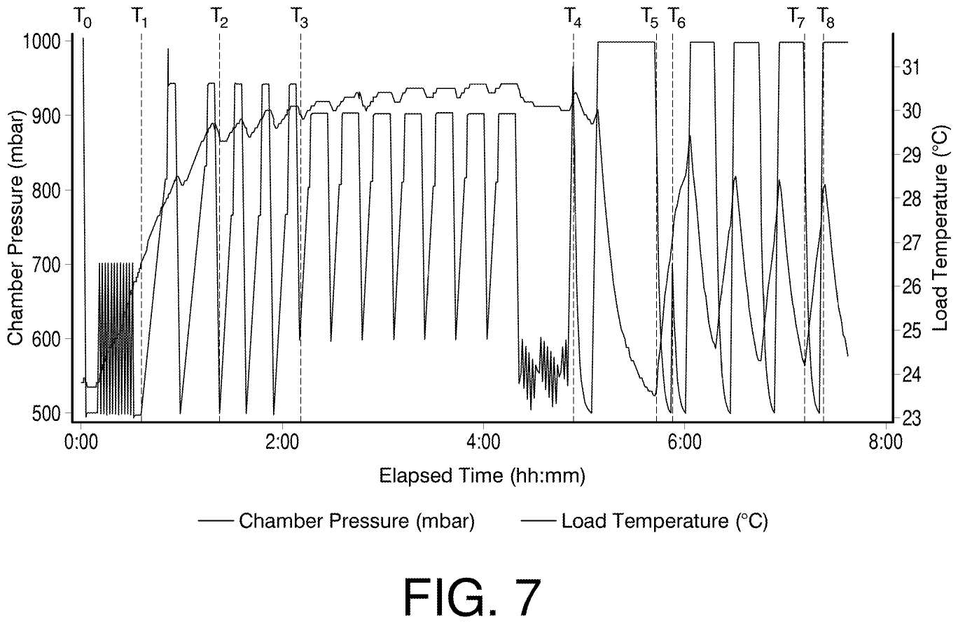

Referring to FIG. 7, the pressure and temperature within the sterilization chamber were plotted as a function of time elapsed, during the performance of Sterilization Method I. The leak test and conditioning steps were performed from time T₀ to time T₁. The primary sterilant exposures were performed from time T₁ to time T₂, the secondary sterilant exposures were performed from time T₂ to time T₃, and the tertiary sterilant exposures were performed from time T₃ to time T₄. The first aeration was performed from time T₄ to time T₅, the second aeration was performed from time T₅ to time T₆, the third, fourth, and fifth aerations were performed from time T₆ to time T₇, the sixth aeration was performed from time T₇ to time T₈, and the seventh aeration was performed from time T₈ to the end of the sterilization method.

After Sterilization Method I was performed, the biological and chemical indicators of Sterilization Load A and Sterilization Load B were analyzed. All biological indicators showed negative growth. Additionally, samples of blister packs were taken from Sterilization Load A and Sterilization Load B, analysis of the sample blister packs demonstrated a six-log reduction in the sterility assurance level.

### Example 3

A sterilization method (Sterilization Method II) was performed on two different sterilization loads, Sterilization Load C and Sterilization Load D, according to the phases and parameters shown in Table 1. Sterilization Method II included two primary sterilant exposures, two secondary sterilant exposures, six tertiary sterilant exposures, and seven aeration pulses. Each primary sterilant exposure had the same parameters as the other primary sterilant exposure, each secondary sterilant exposure had the same parameters as the other secondary sterilant exposure, and each tertiary sterilant exposure had the same parameters as the other tertiary sterilant exposures. Sterilization Load C included eight carts, with each cart including 1,368 prefilled syringes. Sterilization Load D included eight carts, with each cart including 76 prefilled syringes. Each prefilled syringe of Sterilization Load C and Sterilization Load D included about 0.19 grams of liquid medicament (i.e., about 0.18 mL). Each sterilization load also included 30 biological indicators distributed throughout the sterilization load.

After Sterilization Method II was performed, the biological and chemical indicators of Sterilization Load C and Sterilization Load D were analyzed. All biological indicators showed negative growth. Additionally, samples of blister packs were taken from Sterilization Load C and Sterilization Load D, analysis of the sample blister packs demonstrated a six-log reduction in the sterility assurance level.

Sterilization Method II included less secondary sterilant exposures and less tertiary sterilant exposures than Sterilization Method I. As a result, Sterilization Method I included 125 g more of total sterilant exposure than Sterilization Method II. Accordingly, Sterilization II would be expected to be less effective than Sterilization Method I. All biological indicators showing negative growth with Sterilization Method II further evidences that Sterilization Method I is effective at sterilizing the sterilization loads.

### Example 4

After Sterilization Loads A and B were sterilized according to Sterilization Method I, the sterilized prefilled syringes were stored at 25 °C for 6 months. At the time of storage, at 1 month of storage, at 3 months of storage, and at 6 months of storage, the product quality of the prefilled syringes was evaluated to determine if the sterilization method or storage process had an adverse effect on the prefilled syringes. The results of these product quality analyses are summarized in Table 2.

TABLE 2

| | | Results | | | |
|---|---|---|---|---|---|
| | End-of-Shelf-Life | Length of Storage (months) | | | |
| Assay | Acceptance Criteria | t = 0 | 1 | 3 | 6 |
| Appearance | Liquid essentially free from | Pass | Pass | Pass | Pass |
| | visible particulates | | | | |
| Clarity | Not more turbid than | Pass | Pass | Pass | Pass |
| (Turbidity) | Reference Suspension | | | | |
| Color | Not more intensely colored | Pass | Pass | Pass | Pass |
| | than Reference Solution | | | | |
| pH | 5.6-6.1 | 5.9 | 5.9 | 5.9 | 5.9 |
| Total Protein | 103.0-126.0 mg/mL | 113.0 | 114.4 | 113.6 | 115.2 |
| Content (A₂₈₀) | | | | | |
| Potency by | 70%-130% of reference | 113 | 86 | 91 | 79 |
| Bioassay | standard | | | | |
| Purity by | a. ≥91.5% purity | 95.1 | 94.1 | 92.9 | 91.1 |
| Reduced CE-SDS | b. ≤2.5% | 1.2 | 1.6 | 2.1 | 2.6 |
| a. % Purity | c. ≤6.0% | 3.7 | 4.3 | 4.9 | 6.3 |
| b. % R1 | | | | | |
| c. % LMW less R1 | | | | | |
| Purity by Non- | main peak ≥96.5% total area | 99.3 | 98.5 | 98.2 | 96.9 |
| Reduced CE-SDS | | | | | |
| % Main peak | | | | | |
| purity | | | | | |
| Purity by SE-UPLC | a. main peak ≥94.0% total area | 98.0 | 96.3 | 94.5 | 91.9 |
| a. % Main Peak | b. ≤5.0% aggregate | 2.0 | 3.4 | 5.0 | 7.4 |
| b. % Aggregate | | | | | |
| Charged Variant | The test article profile should | Pass | Pass | Pass | Pass |
| Analysis by | be qualitatively similar to the | | | | |
| iCIEF | Reference Standard | | | | |
| a. % Region 1 | electropherogram in terms of | | | | |
| b. % Region 2 | intensity, number, and pattern | | | | |
| c. % Region 3 | of peaks. | | | | |
| | a. 21.5-38.4% | 29 | 29 | 30 | 30 |
| | b. 38.5-50.4% | 45 | 45 | 45 | 46 |
| | c. 13.5-36.4% | 25 | 26 | 0.26 | 0.41 |
| Isoaspartate | ≤0.22 mol isoaspartate/mol | 0.08 | 0.16 | 0.26 | 0.41 |
| | target protein | | | | |
| Polysorbate 20 | 0.022%-0.038% (w/v) | | 0.030 | 0.028 | 0.032 |
| Particulate | ≤50 particles/mL (≥10 μm) | 5 | 5 | 1 | 1 |
| Matter | ≤5 particles/mL (≥25 μm) | 2 | 1 | 0 | 1 |
| (microscopic) | ≤2 particles/mL (≥50 μm) | 0 | 0 | 0 | 0 |
| Maximum | ≤28.2 Newtons (N) | | | | 15.0 |
| Injection | | | | | |
| Force | | | | | |
| Deliverable | 0.07 mL ≤ X < 0.10 mL | | | | 0.086 |
| Volume | | | | | |
| Maximum Use | ≤13 N · cm | | | | ≤13 N · |
| Torque | | | | | cm |

The data from these product quality analyses demonstrate that the sterilization method can achieve sterility acceptance criteria without adverse impact to the drug product.

The present disclosure is further described by the following non-limiting items.

Item 1. A sterilization method comprising:
conducting one or more primary sterilant exposures, wherein each primary sterilant exposure includes:
   adjusting a pressure in a sterilization chamber to a pressure of at least 500 millibars;
   injecting at least 125 grams of vaporized hydrogen peroxide into the sterilization chamber; and
   injecting a first amount of distribution gas, thereby raising the pressure of the sterilization chamber to a pressure of at least 900 millibar;
conducting one or more secondary sterilant exposures, wherein each secondary sterilant exposure includes:
   adjusting the pressure in the sterilization chamber to a pressure of at least 500 millibars;
   injecting at least 50 grams of vaporized hydrogen peroxide into the sterilization chamber; and
   injecting a second amount of distribution gas, thereby raising the pressure of the sterilization chamber to a pressure of at least 900 millibar; and
aerating the sterilization chamber, wherein aerating the sterilization chamber includes:
   adjusting a pressure within the sterilization chamber to a pressure of 450 millibars to 500 millibars; and
   adding an aeration gas to the sterilization chamber, thereby raising the pressure of the sterilization chamber to a pressure of 700 millibars to 1000 millibars.

Item 2. The method of item 1, wherein each primary sterilant exposure includes injecting at least 150 grams of vaporized hydrogen peroxide into the sterilization chamber.

Item 3. The method of item 1, wherein each secondary sterilant exposure includes injecting at least 75 grams of vaporized hydrogen peroxide into the sterilization chamber.

Item 4. The method of item 1, further comprising conducting one or more tertiary sterilant exposures, wherein each tertiary sterilant exposure includes:
adjusting the pressure in the sterilization chamber to a pressure of at least 600 millibars;
injecting at least 50 grams of vaporized hydrogen peroxide into the sterilization chamber; and
injecting a third amount of distribution gas, thereby raising the pressure of the sterilization chamber to a pressure of at least 900 millibar.

Item 5. The method of item 4, wherein the one or more tertiary sterilant exposures comprise at least five tertiary sterilant exposures.

Item 6. The method of item 4, wherein each tertiary sterilant exposure further includes, after injecting the third amount of distribution gas, maintaining a distribution hold for at least 5 minutes.

Item 7. The method of item 1, wherein the one or more primary sterilant exposures include at least two primary sterilant exposures.

Item 8. The method of item 1, wherein the one or more secondary sterilant exposures include at least three secondary sterilant exposures.

Item 9. The method of item 1, wherein each primary sterilant exposure further includes, after injecting the first amount of distribution gas, maintaining a distribution hold for at least 1 minute.

Item 10. A sterilization method comprising:
conditioning a sterilization apparatus comprising a sterilization chamber, wherein conditioning the sterilization apparatus includes at least five conditioning pulses, and each conditioning pulse comprises:
   adjusting a pressure in the sterilization chamber to a conditioning pressure, wherein the conditioning pressure is 450 millibars to 650 millibars;
   adding a conditioning gas to the sterilization chamber, thereby raising the pressure of the sterilization chamber to a pressure that is at least 150 millibar greater than the conditioning pressure;
conducting one or more primary sterilant exposures, wherein each primary sterilant exposure includes:
   adjusting a pressure in the sterilization chamber to a first sterilization pressure;
   injecting at least 125 grams of vaporized hydrogen peroxide into the sterilization chamber; and
   injecting a first amount of distribution gas, thereby raising the pressure of the sterilization chamber to a first transition pressure, wherein the first transition pressure is at least 400 millibar greater than the first sterilization pressure;
conducting one or more secondary sterilant exposures, wherein each secondary sterilant exposure includes:
   adjusting the pressure in the sterilization chamber to a second sterilization pressure;
   injecting at least 50 grams of vaporized hydrogen peroxide into the sterilization chamber; and
   injecting a second amount of distribution gas, thereby raising the pressure of the sterilization chamber to a second transition pressure, wherein the second transition pressure is at least 400 millibar greater than the second sterilization pressure;
conducting one or more tertiary sterilant exposures, wherein each tertiary sterilant exposure includes:
   adjusting the pressure in the sterilization chamber to a third sterilization pressure;
   injecting at least 50 grams of vaporized hydrogen peroxide into the sterilization chamber; and
   injecting a third amount of distribution gas, thereby raising the pressure of the sterilization chamber to a third transition pressure, wherein the third transition pressure is at least 400 millibar greater than the third sterilization pressure.

Item 11. The method of item 10, further comprising:
aerating the sterilization chamber, wherein aerating the sterilization chamber includes:
adjusting a pressure within the sterilization chamber to an aeration pressure, wherein the aeration pressure is at least 400 millibars; and
adding an aeration gas to the sterilization chamber, thereby raising the pressure of the sterilization chamber to an exhaust pressure, wherein the exhaust pressure is at least 700 millibars.

Item 12. The method of item 10, wherein conditioning the sterilization apparatus includes at least ten conditioning pulses.

Item 13. The method of item 10, wherein the total amount of vaporized sterilant injected into the sterilization chamber during the one or more primary sterilant exposures, the one or more secondary sterilant exposures, and the one or more tertiary sterilant exposures is greater than or equal to 800 grams.

Item 14. The method of item 11, wherein:
the aeration pressure is a first aeration pressure and is at least 480 millibars;
the exhaust pressure is a first exhaust pressure and is at least 900 millibars;
aerating the sterilization chamber further includes adjusting a pressure in the sterilization chamber to a second aeration pressure over an exhaust time;
the second aeration pressure is 400 millibars to 550 millibars; and
the exhaust time is at least 25 minutes.

Item 15. The method of item 14, wherein aerating the sterilization chamber further includes adding an aeration gas to the sterilization chamber, thereby raising the pressure of the sterilization chamber to a second exhaust pressure, wherein the second exhaust pressure is at least 900 millibars.

Item 16. The method of item 11, wherein the aeration gas, the distribution gas, and the conditioning gas each have a dew point that is less than or equal to −10 °C.

Item 17. The method of item 10, wherein the one or more primary sterilant exposures includes at least 2 primary sterilant exposures, the one or more secondary sterilant exposures includes at least 3 secondary sterilant exposures, and the one or more tertiary sterilant exposures includes at least 7 tertiary sterilant exposures.

Item 18. A sterilization method comprising:
positioning a sterilization load in a sterilization chamber, wherein a ratio of a liquid mass in the sterilization load to a total mass of the sterilization load is about 0.005 to about 0.02;
conducting one or more sterilant exposures, wherein each sterilant exposure includes:
   adjusting a pressure in the sterilization chamber to a pressure of at least 500 millibars;
   injecting at least 125 grams of vaporized hydrogen peroxide into the sterilization chamber; and
   injecting a first amount of distribution gas, thereby raising the pressure of the sterilization chamber to a pressure of at least 850 millibar; and
aerating the sterilization chamber, wherein aerating the sterilization chamber includes:
   adjusting a pressure within the sterilization chamber to a pressure of at least 400 millibars; and
   adding an aeration gas to the sterilization chamber, thereby raising the pressure of the sterilization chamber to a pressure of at least 700 millibars.

Item 19. The sterilization method of item 18, wherein a ratio of liquid volume in the sterilization load to a total volume of the sterilization load is about 0.0005 to about 0.001.

Item 20. The sterilization method of item 18, the sterilization load includes a plurality of pre-filled delivery devices, and each pre-filled delivery device includes at least 0.15 grams of liquid.

The above description and examples are illustrative, and not intended to be restrictive. For example, and as has been described, the above-described embodiments (and/or aspects thereof) may be used in combination with each other.

What is claimed is:

1. A sterilization method comprising:
conducting one or more primary sterilant exposures, wherein each primary sterilant exposure includes:
adjusting a pressure in a sterilization chamber to a pressure of at least 500 millibars;
injecting at least 125 grams of vaporized hydrogen peroxide into the sterilization chamber; and
injecting a first amount of distribution gas, thereby raising the pressure of the sterilization chamber to a pressure of at least 900 millibar;
conducting one or more secondary sterilant exposures, wherein each secondary sterilant exposure includes:
adjusting the pressure in the sterilization chamber to a pressure of at least 500 millibars;
injecting at least 50 grams of vaporized hydrogen peroxide into the sterilization chamber; and
injecting a second amount of distribution gas, thereby raising the pressure of the sterilization chamber to a pressure of at least 900 millibar; and
aerating the sterilization chamber, wherein aerating the sterilization chamber includes:
adjusting a pressure within the sterilization chamber to a pressure of 450 millibars to 500 millibars; and
adding an aeration gas to the sterilization chamber, thereby raising the pressure of the sterilization chamber to a pressure of 700 millibars to 1000 millibars.

2. The method of claim 1, wherein each primary sterilant exposure includes injecting at least 150 grams of vaporized hydrogen peroxide into the sterilization chamber.

3. The method of claim 1, wherein each secondary sterilant exposure includes injecting at least 75 grams of vaporized hydrogen peroxide into the sterilization chamber.

4. The method of claim 1, further comprising conducting one or more tertiary sterilant exposures, wherein each tertiary sterilant exposure includes:
adjusting the pressure in the sterilization chamber to a pressure of at least 600 millibars;
injecting at least 50 grams of vaporized hydrogen peroxide into the sterilization chamber; and
injecting a third amount of distribution gas, thereby raising the pressure of the sterilization chamber to a pressure of at least 900 millibar.

5. The method of claim 4, wherein the one or more tertiary sterilant exposures comprise at least five tertiary sterilant exposures.

6. The method of claim 4, wherein each tertiary sterilant exposure further includes, after injecting the third amount of distribution gas, maintaining a distribution hold for at least 5 minutes.

7. The method of claim 1, wherein the one or more primary sterilant exposures include at least two primary sterilant exposures.

8. The method of claim 1, wherein the one or more secondary sterilant exposures include at least three secondary sterilant exposures.

9. The method of claim 1, wherein each primary sterilant exposure further includes, after injecting the first amount of distribution gas, maintaining a distribution hold for at least 1 minute.

10. A sterilization method comprising:
conditioning a sterilization apparatus comprising a sterilization chamber, wherein conditioning the sterilization apparatus includes at least five conditioning pulses, and each conditioning pulse comprises:
adjusting a pressure in the sterilization chamber to a conditioning pressure, wherein the conditioning pressure is 450 millibars to 650 millibars;
adding a conditioning gas to the sterilization chamber, thereby raising the pressure of the sterilization chamber to a pressure that is at least 150 millibar greater than the conditioning pressure;
conducting one or more primary sterilant exposures, wherein each primary sterilant exposure includes:
adjusting a pressure in the sterilization chamber to a first sterilization pressure;
injecting at least 125 grams of vaporized hydrogen peroxide into the sterilization chamber; and
injecting a first amount of distribution gas, thereby raising the pressure of the sterilization chamber to a first transition pressure, wherein the first transition pressure is at least 400 millibar greater than the first sterilization pressure;
conducting one or more secondary sterilant exposures, wherein each secondary sterilant exposure includes:
adjusting the pressure in the sterilization chamber to a second sterilization pressure;
injecting at least 50 grams of vaporized hydrogen peroxide into the sterilization chamber; and
injecting a second amount of distribution gas, thereby raising the pressure of the sterilization chamber to a second transition pressure, wherein the second transition pressure is at least 400 millibar greater than the second sterilization pressure;
conducting one or more tertiary sterilant exposures, wherein each tertiary sterilant exposure includes:
adjusting the pressure in the sterilization chamber to a third sterilization pressure;
injecting at least 50 grams of vaporized hydrogen peroxide into the sterilization chamber; and
injecting a third amount of distribution gas, thereby raising the pressure of the sterilization chamber to a third transition pressure, wherein the third transition pressure is at least 400 millibar greater than the third sterilization pressure.

11. The method of claim 10, further comprising:
aerating the sterilization chamber, wherein aerating the sterilization chamber includes:
adjusting a pressure within the sterilization chamber to an aeration pressure, wherein the aeration pressure is at least 400 millibars; and
adding an aeration gas to the sterilization chamber, thereby raising the pressure of the sterilization chamber to an exhaust pressure, wherein the exhaust pressure is at least 700 millibars.

12. The method of claim 10, wherein conditioning the sterilization apparatus includes at least ten conditioning pulses.

13. The method of claim 10, wherein the total amount of vaporized sterilant injected into the sterilization chamber during the one or more primary sterilant exposures, the one or more secondary sterilant exposures, and the one or more tertiary sterilant exposures is greater than or equal to 800 grams.

14. The method of claim 11, wherein:
the aeration pressure is a first aeration pressure and is at least 480 millibars;
the exhaust pressure is a first exhaust pressure and is at least 900 millibars;
aerating the sterilization chamber further includes adjusting a pressure in the sterilization chamber to a second aeration pressure over an exhaust time;
the second aeration pressure is 400 millibars to 550 millibars; and
the exhaust time is at least 25 minutes.

15. The method of claim 14, wherein aerating the sterilization chamber further includes adding an aeration gas to the sterilization chamber, thereby raising the pressure of the sterilization chamber to a second exhaust pressure, wherein the second exhaust pressure is at least 900 millibars.

16. The method of claim 11, wherein the aeration gas, the distribution gas, and the conditioning gas each have a dew point that is less than or equal to −10 °C.

17. The method of claim 10, wherein the one or more primary sterilant exposures includes at least 2 primary sterilant exposures, the one or more secondary sterilant exposures includes at least 3 secondary sterilant exposures, and the one or more tertiary sterilant exposures includes at least 7 tertiary sterilant exposures.

18. A sterilization method comprising:
positioning a sterilization load in a sterilization chamber, wherein a ratio of a liquid mass in the sterilization load to a total mass of the sterilization load is about 0.005 to about 0.02;
conducting one or more sterilant exposures, wherein each sterilant exposure includes:
adjusting a pressure in the sterilization chamber to a pressure of at least 500 millibars;
injecting at least 125 grams of vaporized hydrogen peroxide into the sterilization chamber; and
injecting a first amount of distribution gas, thereby raising the pressure of the sterilization chamber to a pressure of at least 850 millibar; and
aerating the sterilization chamber, wherein aerating the sterilization chamber includes:
adjusting a pressure within the sterilization chamber to a pressure of at least 400 millibars; and
adding an aeration gas to the sterilization chamber, thereby raising the pressure of the sterilization chamber to a pressure of at least 700 millibars.

19. The sterilization method of claim 18, wherein a ratio of liquid volume in the sterilization load to a total volume of the sterilization load is about 0.0005 to about 0.001.

20. The sterilization method of claim 18, the sterilization load includes a plurality of pre-filled delivery devices, and each pre-filled delivery device includes at least 0.15 grams of liquid.
